(19) United States
(12) Patent Application Publication
(10) Pub. No.: US 2018/0117076 A1
(43) Pub. Date: May 3, 2018

(54) MiRNA compositions for the treatment of mature B-cell neoplasms

(71) Applicant: FUNDACIÓN CENTRO NACIONAL DE INVESTIGACIONES CARDIOVASCULARES CARLOS III (CNIC), Madrid (ES)

(72) Inventors: Almudena RODRÍGUEZ RAMIRO, Madrid (ES); Nahikari BARTOLOMÉ IZQUIERDO, Madrid (ES); Virginia GARCÍA DE YÉBENES, Madrid (ES)

(21) Appl. No.: 15/573,394

(22) PCT Filed: May 13, 2016
(86) PCT No.: PCT/EP2016/060927

§ 371 Date: Nov. 10, 2017

(30) Foreign Application Priority Data
May 14, 2015 (EP) 15382249.9

(51) Int. Cl. A61K 31/7105; A61K 31/713; C12N 15/113; C12N 15/867; A61P 35/00; A61K 9/00

(52) U.S. Cl. CPC A61K 31/7105; A61K 31/713; C12N 15/113; C12N 15/867; C12N 2330/51; A61K 9/0019; C12N 2310/141; C12N 2320/32; A61P 35/00

(57) ABSTRACT

Provided herein are compositions comprising miR-28, including compositions comprising miRNA-28 expression vectors, as well as compositions comprising compounds that mimic the miRNA activity of miR-28. In certain embodiments, the compositions comprise oligomeric compounds comprising oligonucleotides having nucleobase sequence identity to miR-28. In certain embodiments, the nucleobase sequence of an oligonucleotide having identity to a miR-28 comprises a seed region of the miRNA-28. The compositions may comprise one or more lipids. In certain embodiments, the one or more lipids are selected from a cationic lipid, a neutral lipid, a sterol, and a disaggregation lipid. Also provided herein are methods for the treatment of mature B-cell neoplasms, comprising administering to a subject having a mature B-cell neoplasm a composition comprising miR-28, including compositions comprising miRNA-28 expression vectors, and/or a composition comprising a compound that mimics the miRNA activity of miRNA-28.

### FIELD OF THE INVENTION

Provided herein are compositions and methods for their use in the treatment of mature B-cell neoplasias, in particular mature B-cell neoplasias derived from germinal centre (GC) B cells.

### BACKGROUND OF THE INVENTION

The vast majority of lymphoid neoplasias are originated from lymphocytes that have germinal center experience, and include, among others, Burkitt lymphoma (BL), diffuse large B cell lymphoma (DLBCL), follicular lymphoma (FL) and chronic lymphocytic leukemia (CLL). It is estimated that about 60.000-80.000 new cases of germinal-center derived neoplasias are diagnosed every year in Europe alone.

While a significant fraction of aggressive B cell lymphomas can be cured with current therapies, most commonly doxorubicin-based combination chemotherapy with rituximab (R-CHOP), these are often highly intensive treatments, requiring admission to the hospital. In addition, almost half of the DLBCL and BL cases are resistant to these therapeutic approaches, or relapse within 5 years of treatment (LSS).

### BRIEF DESCRIPTION OF THE INVENTION

We herein propose the use of miR-28 as a new therapeutic agent for lymphoid neoplasias originated from lymphocytes that have germinal center experience. Our results show that miR-28 expression impairs tumour cell proliferation and promotes tumour cell death. In addition, lentiviral delivery of miR-28 impairs tumour growth in xenograft models of BL and ABC-DLBCL (a subtype of DLBCL particularly refractory to conventional treatment). Likewise, as shown herein, lentiviral delivery of miR-28 regresses established BL tumors. Moreover, we herein show that compositions of synthetic miR-28 sequence (microRNA mimic) display anti-tumoral activity in BL xenografts both when delivered intra-tumorally or intravenously.

Finally, and unexpectedly, miR-28 shows negligible toxicity as illustrated in the assays performed herein. In summary, the data presented in this specification shows that miR-28 can have a direct clinical application for lymphoma therapy.

Thus, provided herein are compositions comprising miR-28, including compositions comprising miRNA-28 expression vectors, as well as compositions comprising compounds that mimic the miRNA activity of miR-28. In certain embodiments, the compositions comprise oligomeric compounds comprising oligonucleotides having nucleobase sequence identity to miR-28. In certain embodiments, the nucleobase sequence of an oligonucleotide compound having identity to a miR-28 comprises a seed region of the miRNA-28. The compositions may comprise one or more lipids. In certain embodiments, the one or more lipids are selected from a cationic lipid, a neutral lipid, a sterol, and a disaggregation lipid.

Also provided herein are methods for the treatment of mature B-cell neoplasms, comprising administering to a subject having a mature B-cell neoplasm a composition comprising miR-28, including compositions comprising miRNA-28 expression vectors, and/or a composition comprising a compound that mimics the miRNA activity of miRNA-28. Such methods may, for example, comprise the administration of a composition comprising an oligomeric compound consisting of an oligonucleotide, wherein the oligonucleotide has nucleobase sequence identity to miR-28. The mature B-cell neoplasia may be any type of B-cell neoplasia, for example, follicular lymphoma, diffuse large B cell lymphoma (DLBCL), Burkitt's lymphoma and chronic lymphocytic leukemia (CLL), all derived from germinal centre (GC) B cells. Thus, in a particular embodiment of the present invention, the mature B-cell neoplasia is derived from germinal centre (GC) B cells.

Provided herein are compositions comprising an oligomeric compound comprising an oligonucleotide consisting of 7 to 30 linked nucleosides, wherein the nucleobase sequence of the oligonucleotide has at least 80% seed region identity with the nucleobase sequence of miR-28. Provided herein are compositions comprising an oligomeric compound comprising an oligonucleotide hybridizing to a complementary nucleotide sequence, wherein the oligonucleotide has at least 80% seed region identity with the nucleobase sequence of miR-28 and the nucleobase sequence of the complementary oligonucleotide has at least 80% complementarity to the oligonucleotide.

In certain embodiments, the oligomeric compound consists of the oligonucleotide hybridized to a complementary oligonucleotide.

In certain embodiments, the nucleobase sequence of the oligonucleotide has at least 85%, at least 90%, or at least 95% seed region identity with the nucleobase sequence of miR-28. In certain embodiments, the nucleobase sequence of the oligonucleotide has 100% seed region identity with the nucleobase sequence of miR-28. In certain embodiments, the oligonucleotide has at least 70%, at least 75%, at least 80%, at least 90%, or at least 95% overall identity with the nucleobase sequence of miR-28. In certain embodiments, the oligonucleotide has 100% overall identity with the nucleobase sequence of miR-28.

In certain embodiments, the miR-28 comprises SEQ ID NO: 1:

(gguccuugcccucaaggagcucacagucuauugaguuaccuuucugacu
uucccacuagauugugagcuccuggagggcaggcacu).

In certain embodiments, the miR-28 consists of SEQ ID NO: 1:

(gguccuugcccucaaggagcucacagucuauugaguuaccuuucugacu
uucccacuagauugugagcuccuggagggcaggcacu).

In certain embodiments, the miR-28 comprises SEQ ID NO: 2:

(GGUCCUUGCCCUCAAGGAGCUCACAGUCUAUUGAGUUACCUUUCUGACU
UUCCCACUAGAUUGUGAGCUCCUGGAGGGCAGGCACU).

In certain embodiments, the miR-28 consists of SEQ ID NO: 2:

(GGUCCUUGCCCUCAAGGAGCUCACAGUCUAUUGAGUUACCUUUCUGACU
UUCCCACUAGAUUGUGAGCUCCUGGAGGGCAGGCACU).

In certain embodiments, the miR-28 compounds described herein comprises at least one modified sugar or comprises a plurality of modified sugars. In certain embodiments, each nucleoside of the oligonucleotide comprises a modified sugar. In certain embodiments, the complementary oligonucleotide comprises at least one modified sugar or comprises a plurality of modified sugars. In certain embodiments, each nucleoside of the complementary oligonucleotide comprises a modified sugar. In certain embodiments, the modified sugar is independently selected from 2′-O-methyl, 2′-O-methoxyethyl, 2′-fluoro, and a bicyclic sugar.

In certain embodiments, the miR-28 compounds described herein comprises at least one modified internucleoside linkage. In certain embodiments, the oligonucleotide comprises a plurality of modified internucleoside linkages. In certain embodiments, the complementary oligonucleotide comprises at least one modified internucleoside linkage. In certain embodiments, the complementary oligonucleotide comprises a plurality of modified internucleoside linkages. In certain embodiments, each internucleoside linkage is a modified internucleoside linkage. In certain embodiments, the modified internucleoside linkage is phosphorothioate.

In certain embodiments, the miR-28 compounds described herein comprises at least one modified nucleobase. In certain embodiments, the complementary oligonucleotide comprises at least one modified nucleobase. In certain embodiments, the modified nucleobase is a 5-methylcytosine.

Provided herein are compositions comprising any of the miR-28 compounds described herein comprising an oligonucleotide consisting of 7 to 30 linked nucleosides, wherein the nucleobase sequence of the oligonucleotide has at least 80% seed region identity with the nucleobase sequence of miR-28, and at least one, at least two, at least three, or at least four lipids. In certain embodiments, a lipid is a cationic lipid. In certain embodiments, a lipid is an amino lipid. In certain embodiments, a lipid is a sterol. In certain embodiments, a lipid is a disaggregation lipid. In certain embodiments, a lipid is a neutral lipid. In certain embodiments, each lipid is selected from among a cationic lipid, a neutral lipid, a sterol, and a disaggregation lipid. In certain embodiments, the cationic lipid is 2,2-Dilinoleyl-4-dimethylaminoethyl-[1,3]-dioxolane. In certain embodiments, the sterol is cholesterol. In certain embodiments, the disaggregation lipid is a polyethylene glycol lipid (PEG-Lipid). In certain embodiments, the PEG-lipid is PEG-didimyristoyl glycerol (PEG-DMG). In certain embodiments, the PEG-lipid is PEG-distyryl glycerol (PEG-DSG). In certain embodiments, the PEG-lipid is PEG-carbamoyl-1,2-dimyristyloxypropylamine (PEG-cDMA). In certain embodiments, the lipid is a phospholipid. In certain embodiments, the phospholipid is phosphatidylcholine. In certain embodiments, the phosphatidylcholine is distearoylphosphatidylcholine. In certain embodiments, the phosphatidylcholine is dipalmitoylphosphatidylcholine. In certain embodiments, the composition comprises a cationic lipid, neutral lipid, sterol, and disaggregation lipid in a molar ratio of 50 to 60:7 to 10:30 to 40:1 to 5. In certain embodiments, the molar ratio is 57.5:7.5:31.5:3.5; 60:7.5:31:1.5; or 50:10:38.5:1.5.

In certain embodiments, the lipid:oligomeric compound ratio is from 5 to 35 or from 5 to 15. In certain embodiments, the lipid:oligomeric compound ratio is 6; 7; 8; 9; 10; or 11.

Any of the compositions provided herein may comprise a pharmaceutically acceptable carrier or diluent.

Provided herein are methods for treating mature B-cell neoplasias, comprising administering to a subject having a mature B-cell neoplasia a composition provided herein. The mature B-cell neoplasia may be any type of B-cell neoplasia, for example, follicular lymphoma, diffuse large B cell lymphoma (DLBCL), Burkitt's lymphoma and chronic lymphocytic leukemia (CLL), all derived from germinal centre (GC) B cells. Thus, in a particular embodiment of the present invention, the mature B-cell neoplasia is derived from germinal centre (GC) B cells. The subject may be a human. The route of administration may comprise intravenous administration, subcutaneous administration, intratumoral administration, or chemoembolization.

In certain embodiments, the methods provided herein comprise at least one additional therapy. The at least one additional therapy may comprise a chemotherapeutic agent and/or radiation therapy. The chemotherapeutic agent may be selected from 5-fluorouracil, gemcitabine, doxorubicine, mitomycin c, sorafenib, etoposide, carboplatin, epirubicin, irinotecan and oxaliplatin. The at least one additional therapy may be administered at the same time, less frequently, or more frequently than administration of a composition provided herein.

In any of the methods provided herein, the composition may be administered once per day, once per week, once per two weeks, once per three weeks, or once per four weeks.

In any of the methods provided herein, administering results in the inhibition of tumour cell proliferation and the promotion of tumour cell death. Thus, in any of the methods provided herein, the administering prevents an increase in tumour size and/or an increase in tumour number. The administering may prevent, stop or slow metastatic progression. The administering may extend the overall survival time of the subject. The administering may extend progression-free survival of the subject.

The present invention also provides for any of the compounds described herein for use as a medicament. The present invention also provides for any of the compounds described herein for preventing, treating, or diagnosing any of the diseases or conditions described herein. The present invention also provides for use of any of the compounds described herein for preventing, treating, or diagnosing any of the diseases or conditions described herein.

These and other embodiments of the present invention will become apparent in conjunction with the figures, description and claims that follow.

### BRIEF DESCRIPTION OF THE FIGURES

FIG. 1. miR-28 negatively regulates the germinal center reaction. A) miR-28 expression was assessed by qRT-PCR in naïve B cells (CD19+Fas−GL7−IgA−), germinal center B cells (GC, CD19+Fas+GL7+), and switched B cells (post−GC, CD19+Fas−GL7−IgA+) from Peyer's patches (left) (n=2) and also in spleen GC B cells (B220+Fas+GL7+) at the indicated days after immunization of C57/BL mice with sheep red blood cells (SRBC) (right) (n=2). B) Spleen B cells were stimulated with LPS and IL4 and transduced with control or pre-miR-28-containing retroviral constructs. CSR to IgG1 was assessed by FACS analysis 3 days after transduction. Left, representative FACS plot; right, quantification. C) miR-28-sponge (miR-28 SPG) retroviral construct for miR-28 inhibition. Four miR-28 complementary sites (28-T), separated by 4-nt spacers, were placed downstream of GFP in the MGP vector. D-E) Mouse chimeras transplanted with bone marrow cells transduced with miR-28 SPG or empty vector were analyzed by FACS 10-14 days after immunization. Representative plots of GC (top), switched (middle) and plasma (bottom) B cells are shown in D) and quantifications in E). Each symbol represents an individual mouse. PBS: non-immunized chimeric mice. Data are from 3 independent transplantation and immunization experiments, and are normalized to PBS values; *p<0.05, unpaired t test.

FIG. 2. Identification of miR-28 targets in lymphoma B cells. A) miR-28 microarray expression data (GSE29193) in cohorts of patients with chronic lymphocytic leukemia (CLL, 18 samples), diffuse large B cell lymphoma (DLBCL, 29 samples), Burkitt lymphoma (BL, 12 samples), and follicular lymphoma (FL, 23 samples). Controls were human naive B cells (CD19+IgD+CD27−) (3 samples, filled circles) and GC B cells (CD10+CD19+) (4 samples, open circles) extracted from tonsils of healthy donors. Adjusted p values were calculated with the Benjamini and Hochberg method (FDR). B) Ramos BL cells were transduced with scramble or miR-28 vectors, and RFP+ cells were analyzed by RNA-Seq and iTRAQ for differential transcriptome and proteome characterization, respectively. Plots are volcano representations of transcriptomic and proteomic changes in Ramos cells upon miR28 re-expression. Dots represent mean fold change (miR-28 Ramos cells/control Ramos cells) at the level of transcripts (RNA-Seq; 6 replicates) or proteins (iTRAQ; 4 replicates) (X-axes) against the statistical significance of the change (Y-axes), both in 2-base logarithmic scale. Changes were considered significant at cut-offs of p<0.05 (RNA26 Seq) or FDR<10% (p<0.0038) (iTRAQ) (red lines). C) GSEA of predicted miR-28 targets versus transcriptome analysis of miR-28-expressing BL cells. D) q-RT-PCR validation of miR-28-mediated transcript downregulation of six representative genes found to be significantly downregulated in the RNAseq analysis (*p <0.05, unpaired t test). E) miR-28-altered transcripts (upper graph) and proteins (lower graph) were annotated with Functional Gene Ontology categories. X axes plot the number of miR-28-altered transcripts or proteins within each GO category; Y axes plot the proportion of miR-28-altered transcripts or proteins within each GO category. Circle area represents the relative contribution of each GO category to the total number of miR-28-altered transcripts or proteins. F) The graph shows the cumulative distributions of the standardized variable at the protein level (Zqa) plotted separately for each miR-28-altered category in the iTRAQ proteomic analysis. G) Ingenuity Pathway Analysis of the proteins differentially expressed upon miR-28 expression in BL cells (BCR signaling pathway enrichment, p=10-46). Upregulated proteins are depicted in green, downregulated in red, and nodes in blue.

FIG. 3. miR-28 regulates proliferation and cell death in lymphoma B cells by dampening the BCR signaling pathway. A) Extracts of RFP+ Ramos BL cells, transduced with control or pre-miR-28-containing retroviral constructs, were immunoblotted with antibodies to phospho-ERK1/2 (T202/Y204), ERK1/2, and phospho-AKT (S473). Numbers beneath the bands show protein quantification after normalization to the α-tubulin loading control signal. Bar graphs on the right show data from two independent experiments. B) AKT phosphorylation was measured by flow cytometry after anti-IgM stimulation of RFP+ Ramos cells expressing miR-28 (shaded histogram) or scramble RNA (open histogram). The panel shows representative flow cytometry plots (left) and quantification of 4 independent experiments (right). MFI, mean fluorescence intensity. *p<0.05, unpaired t test. C) q-RT-PCR of Bc1-2, NFKB2, and IKKB in miR-28 versus control Ramos RFP+ BL cells (n=3). D) Graphs show miR-28 expression plotted against transcript levels of NFKB2, IKKB and BCL2 in human primary ABC DLBCL lymphoma cohorts (data extracted from (Iqbal et al., 2015)). E) The MD-901 ABC-DLBCL cell line and the Raji and Ramos BL cell lines were transduced with pTRIPZ vectors encoding miR-28 (solid circles) or scramble RNA (open circles). RFP+ cells were cultured and counted every day throughout the culture period. Data are from at least two independent experiments. *, p<0.05, unpaired t test. F) Primary splenic B cells were labeled with violet cell tracer, transduced with miR-28 or an empty control retroviral vector, and cultured in vitro with LPS+IL4. The left panel shows representative FACS histograms 2 days after retroviral transduction (open line, control; shaded histogram, miR-28). PI: proliferation index. The right panel shows quantification of the proportion of cells that have undergone 0 to 5 divisions. G) FACS analysis of cell cycle in RFP+ miR-28- or control transduced Ramos BL cells labeled with propidium iodide. Cell cycle phases are quantified in the graph plot. H) FACS analysis of BrdU incorporation in RFP+ miR-28-and controltransduced Ramos BL cells. Quantification is shown on the right. I-J) 7AAD and annexin V staining (n=2) (I) and active caspase 3 staining (n=6) (J) in RFP+ miR-28-and controltransduced Ramos BL cells.

FIG. 4. miR-28 does not show toxicity in mouse fibroblasts or human T cell lines. NIH-3T3 mouse fiblroblast cell line (A) and Jurkat T cell line (B) were transduced with pTRIPZ inducible lentiviral vectors containing the miR-28 precursor sequence (solid circles) or a scrambled sequence as negative control (open circles). After transduction cells were selected with antibiotic, induced in the presence of doxycycline for two days and RFP+ cells were isolated by preparative flow cytometry. Cells were cultured in complete medium and cell number was calculated everyday throughout the culture.

FIG. 5. Schematics of the therapeutic protocols used in this study. A) Xenograft tumor establishment. Used for the experiments shown in FIGS. 2E, F, G, H, I. B) Xenograft tumor regression. Used for the experiments shown in FIGS. 3A and B. C) Xenograft tumor treatment with intratumoral miR-28 mimic. Used for the experiments shown in FIGS. 3C, D, F, and G. D) Xenograft tumor treatment with intravenous miR-28 mimic administration. Used for the experiments shown in FIG. 3E. E) Primary mouse lymphoma treatment with intravenous miR-28 mimic administration. Used for the experiments shown in 3H.

FIG. 6. miR-28 expression impairs B cell lymphoma growth in vivo. A) Tumor volume in NGS mice injected with Ramos BL cells expressing miR-28 or scramble RNA (control). Ramos BL cells were transduced with pTRIPZ vectors encoding miR-28 precursor sequence (blue) or scrambled control sequence and induced with doxycycline. RFP+ cells isolated by flow cytometry were injected subcutaneously into either lank of NSG mice. Doxycycline was administered in the drinking water a week before injection and throughout the experiment.

Tumors were measured at the indicated times and volume was calculated as volume [mm3]=(width [mm])2×(length [mm])/2. Each circle represents an individual tumor. B) Ramos xenografts were established as described in A) and tumor growth critical mass (tumor burden<20 mm in any of the three dimensions) was used as a measure of survival for Kaplan-Mayer representation. C) Representative images of miR-28 or control xenograft tumors at endpoint (26 days post injection). D) Weight of miR-28 or control xenograft tumors at endpoint (26 days post injection). E) Ramos xenografts were prepared as described in A). mice were sacrificed at 26 days post injection and tumors were stained with anti-Ki67, anti-caspase-3, and anti-Bc1-2. Left panels show representative micrographs. Images were acquired at 20× (Ki67 and caspase 3; 40× in insets) or 40× (Bc12). Scale bars, 100 μm (Ki67) and 50 □m (Bc1-2). Right, quantification of caspase-3 and Bc1-2 staining. *, p<0.05; ***p<0.001, unpaired t test. F) Tumor volume in NGS mice injected with MD-901 ABC-DLBCL or Raji BL cells expressing miR-28 or scramble RNA (control). MD-901 ABC-DLBCL and Raji BL cells were transduced with pTRIPZ vectors encoding miR-28 precursor sequence (blue bars) or scrambled control sequence (white bars) and induced with doxycycline.

FIG. 7. miR-28 expression suppresses established human lymphomas. A) Ramos BL cells were transduced with lentivirus encoding miR-28 or scramble RNA (control), and cells were injected subcutaneously, without induction, into NSG mice. Xenografts were left to establish for 21 days (until reaching ˜250 mm3), and miR-28 expression was then induced by doxycycline in the drinking water. Graphs show volumes of individual tumors and mean values at the indicated times before (−Dox) and after (+Dox) doxycycline administration. B) Tumor weights of miR-28 and control xenografts at 18 days post Dox treatment. C) Intratumor administration with synthetic miR-28 mimic suppresses established BL tumors. Wild type Ramos cells were injected subcutaneously into NSG mice; after xenografts were established (tumor volume >200 mm3), synthetic miR-28 mimic (green bars) or scrambled control mimics (white bars) were administered intratumorally. Graphs show tumor volumes at the indicated times in xenografts before and after treatment with 0.1 nmol (left) or 0.5 nmol (right) of miRNA mimic. D) Endpoint-to-pretreatment volume ratios for the xenografts in C. E) Ramos xenografts were established as in C and mice were treated by intravenous administration of 7 nmol miR-28 mimic (green) or scrambled mimics (control, filled circles). The graph shows tumor volumes at the indicated times. Each circle corresponds to an independent tumor. *, p<0.05; **p<0.01, unpaired t test.

FIG. 8. miR-28 expression suppresses established primary lymphomas. A) Representative flow cytometry plots of lymph nodes from control and λ-MYC littermate mice. B) Representative histochemistry images of control and λ-MYC littermate spleens. C) qRT-PCR analysis of miR-28 in naïve, GC, and lymphoma cells from λ-MYC mice. D) Cells from enlarged spleen or lymph nodes of λ-MYC mice were injected subcutaneously into NSG mice. Once tumors were detectable (>200 mm3), miR-28 (green) or control (white bars, black circles) mimics were administered intratumorally (FIG. S2E) in 3 injections. Tumor growth was monitored throughout the experiment (left graph, arrows indicate mimic injections) and at endpoint (central graph and image). E-F). Lymph node cells from control and λ-MYC littermate mice were injected intravenously into NSG recipients. E) Spleen weights of NSG mice sacrificed at the indicated post-transplant times. F) Representative flow cytometry analysis of bone marrow or spleen from NSG mice 10 days after transplant with control or λ-MYC cells. Representative spleens are shown to the right. G-H) Cells from enlarged λ-MYC spleen or lymph nodes were injected intravenously into NSG recipient mice; 10 days later mice received intravenous injections of miR-28 or control mimics. G) Spleen weights of transplanted NGS mice (left) and representative images (right) after treatment with miR-28 or control mimics. H) Proportion of spleen B cells in transplanted NSG mice after treatment with miR-28 or control mimics. I) Spleens from NSG mice transplanted with λ-MYC and treated with control or miR-28 mimic were stained with the indicated antibodies. Scale bar 100 μm for Pax5 and Ki67 and 25 μm for caspase 3. Quantification of caspase 3 staining is shown on the right.

### DESCRIPTION OF THE INVENTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sangvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can command go, but equivalent information can be found by searching the internet. Reference thereto evidences the availability and public dissemination of such information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Mature B-cell neoplasms (or neoplasia)” means clonal proliferations of B cells at various stages of differentiation, ranging from näive B cells to mature plasma cells, in particular neoplasms arising from a restricted stage of differentiation, namely, germinal center. Mature B-cell neoplasms that are originated from lymphocytes that have germinal center experience, include, among others, Burkitt lymphoma (BL), diffuse large B cell lymphoma (DLBCL), preferably of the ABC subtype, follicular lymphoma (FL) and chronic lymphocytic leukemia (CLL).

“Subject” means a human or non-human animal selected for treatment or therapy.

“Subject in need thereof” means a subject identified as in need of a therapy or treatment.

“Administering” means providing a pharmaceutical agent or composition to a subject, and includes, but is not limited to, administering by a medical professional and self-administering.

“Parenteral administration,” means administration through injection or infusion. Parenteral administration includes, but is not limited to, subcutaneous administration, intravenous administration, or intramuscular administration.

“Subcutaneous administration” means administration just below the skin.

“Intravenous administration” means administration into a vein.

“Intratumoral administration” means administration within a tumour.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects need only be overlapping for a period of time and need not be coextensive.

“Chemoembolization” means a procedure in which the blood supply to a tumor is blocked surgically, mechanically, or chemically and chemotherapeutic agents are administered directly into the tumor.

“Duration” means the period of time during which an activity or event continues. In certain embodiments, the duration of treatment is the period of time during which doses of a pharmaceutical agent or pharmaceutical composition are administered.

“Therapy” means a disease treatment method.

“Treatment” means the application of one or more specific procedures used for the cure or amelioration of a disease. In certain embodiments, the specific procedure is the administration of one or more pharmaceutical agents.

“Amelioration” means a lessening of severity of at least one indicator of a condition or disease.

In certain embodiments, amelioration includes a delay or slowing in the progression of one or more indicators of a condition or disease. The severity of indicators may be determined by subjective or objective measures which are known to those skilled in the art.

“Prevention” refers to delaying or forestalling the onset or development or progression of a condition or disease for a period of time, including weeks, months, or years.

“Prevent the onset of” means to prevent the development a condition or disease in a subject who is at risk for developing the disease or condition. In certain embodiments, a subject at risk for developing the disease or condition receives treatment similar to the treatment received by a subject who already has the disease or condition.

“Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

“Therapeutic agent” means a pharmaceutical agent used for the cure, amelioration or prevention of a disease.

“Overall survival time” means the time period for which a subject survives after diagnosis of or treatment for a disease.

“Progression-free survival” means the time period for which a subject having a disease survives, without the disease getting worse. In certain embodiments, progression-free survival is assessed by staging or scoring the disease.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

“Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

“Therapeutically effective amount” refers to an amount of a pharmaceutical agent that provides a therapeutic benefit to an animal.

“Pharmaceutical composition” means a mixture of substances suitable for administering to an individual that includes a pharmaceutical agent. For example, a pharmaceutical composition may comprise an oligonucleotide and a sterile aqueous solution.

“Pharmaceutical agent” means a substance that provides a therapeutic effect when administered to a subject.

“Active pharmaceutical ingredient” means the substance in a pharmaceutical composition that provides a desired effect.

“Expression” means any functions and steps by which a gene's coded information is converted into structures present and operating in a cell.

“5′ target site” refers to the nucleobase of a target nucleic acid which is complementary to the 5′-most nucleobase of a particular oligonucleotide.

“3′ target site” means the nucleobase of a target nucleic acid which is complementary to the 3′-most nucleobase of a particular oligonucleotide.

“Region” means a portion of linked nucleosides within a nucleic acid.

“Segment” means a smaller or sub-portion of a region.

“Nucleobase sequence” means the order of contiguous nucleobases, in a 5′ to 3′ orientation, independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding.

“Complementary” means that an oligomeric compound is capable of hybrizing to a target nucleic acid under stringent hybridization conditions.

“Complementarity” means the nucleobase pairing ability between a first nucleic acid and a second nucleic acid.

“Fully complementary” means each nucleobase of an oligomeric compound is capable of pairing with a nucleobase at each corresponding position in a target nucleic acid.

“Percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound. In certain embodiments, percent complementarity of an means the number of nucleobases that are complementary to the target nucleic acid, divided by the length of the modified oligonucleotide.

“Overall identity” means the number of nucleobases in a first oligomeric compound that are identical to nucleobases at corresponding positions in a second oligomeric compound, divided by the total number of nucleobases in the first oligomeric compound.

“Region identity” means the number of nucleobases in a region of a first oligomeric compound that are identical to nucleobases at corresponding positions in a second oligomeric compound, divided by the number of nucleobases in the region.

“Central complementary region” means a region of complementarity between a first oligonucleotide and a second oligonucleotide, where the hybridization of the first and second oligonucleotide results in the formation of one or more overhangs.

“Seed region identity” means the nucleobase sequence identity between the nucleobase sequence of a seed region and contiguous nucleobases of an oligomeric compound. “Seed region identity” can also be referred to as “seed sequence identity.”

“Nucleobase identity” means nucleobases that are the same as one another.

“Nucleobase sequence identity” means nucleobase sequences that are at least partially the same as one another. Nucleobase sequence identity may be less than 100%.

“Hybridize” means the annealing of complementary nucleic acids that occurs through nucleobase complementarity.

“Mismatch” means a nucleobase of a first nucleic acid that is not capable of pairing with a nucleobase at a corresponding position of a second nucleic acid.

“Non-identical nucleobase” means nucleobases that are different from one another.

“Non-complementary nucleobase” means two nucleobases that are not capable of pairing through hydrogen bonding.

“MicroRNA” means an endogenous non-coding RNA, which is the product of cleavage of a pre-miRNA by the enzyme Dicer. Examples of mature miRNAs are found in the miRNA database known as miRBase (available on the world wide web at “microrna.sanger.ac.uk/”). In certain embodiments, microRNA is abbreviated as “miRNA” or “miR.”

“miR-28 family” means miR-28 microRNA or microRNA precursors that share a seed sequence.

“miR-28” means a mature microRNA that is a member of the miR-28 family. In particular, the present invention is concern with synthetic miR-28 or “miR-28 mimic” having the nucleobase sequence of SEQ ID NO: 1. In certain embodiments, the miR-28 mimic comprises SEQ ID NO: 1:

(gguccuugcccucaaggagcucacagucuauugaguuaccuuucugacu
uucccacuagauugugagcuccuggagggcaggcacu).

In certain embodiments, the miR-28 mimic consists of SEQ ID NO: 1:

(gguccuugcccucaaggagcucacagucuauugaguuaccuuucugacu
uucccacuagauugugagcuccuggagggcaggcacu).

In certain embodiments, the present invention is concern with a vector (such as a lentiviral vector) comprising a miR-28 which in turn comprises or consists of SEQ ID NO: 2:

(GGUCCUUGCCCUCAAGGAGCUCACAGUCUAUUGAGUUACCUUUCUGACU
UUCCCACUAGAUUGUGAGCUCCUGGAGGGCAGGCACU).

“Mimic” means an oligomeric compound comprising an oligonucleotide having nucleobase sequence identity to a mature.

“miR-28 mimic” means an oligomeric compound comprising an oligonucleotide having nucleobase sequence identity to miR-28.

“Seed sequence” or “seed region” means a nucleobase sequence comprising from 6 to 8 contiguous nucleobases of nucleobases 1 to 8 of the 5′-end of a mature microRNA-28 sequence.

“Seed sequence” and “seed region” can be used interchangeably and refer to the same sequence as it is defined for the term “seed sequence.”

“Seed match sequence” means a nucleobase sequence that is complementary to a seed sequence, and is the same length as the seed sequence.

“Seed-matched transcript” means a transcript that contains a nucleobase sequence to which a seed sequence is complementary. In certain embodiments, the expression of a seed-matched transcript is regulated by a microRNA comprising the seed sequence that is complementary to the seed-matched transcript.

“Oligomeric compound” means a compound comprising a polymer of linked monomeric subunits. In certain embodiments, an oligomeric compound is a single-stranded oligomeric compound. In certain embodiments, an oligomeric compound is a double-stranded oligomeric compound. “Oligonucleotide” means a polymer of linked nucleosides, each of which can be modified or unmodified, independent from one another.

“Naturally occurring internucleoside linkage” means a 3′ to 5′ phosphodiester linkage between nucleosides.

“Natural sugar” means a sugar found in DNA (2′-H) or RNA (2′-OH).

“Natural nucleobase” means a nucleobase that is unmodified relative to its naturally occurring form.

“Internucleoside linkage” means a covalent linkage between adjacent nucleosides.

“Linked nucleosides” means nucleosides joined by a covalent linkage.

“Nucleobase” means a heterocyclic moiety capable of non-covalently pairing with another nucleobase.

“Modified oligonucleotide” means an oligonucleotide having one or more modifications relative to a naturally occurring terminus, sugar, nucleobase, and/or internucleoside linkage.

### DETAILED DESCRIPTION OF THE INVENTION

It is demonstrated herein that compositions comprising a miR-28 mimic or a vector (such as a viral vector) comprising miR-28 can be efficiently administered to a subject suffering from a mature B-cell neoplasm and results in the inhibition of tumour cell proliferation and the promotion of tumour cell death. The administering may also prevent, stop or slow metastatic progression. The administering may extend the overall survival time of the subject. The administering may extend progression-free survival of the subject.

Accordingly, provided herein are compositions and methods for the treatment of mature B-cells neoplasms. Also provided herein are pharmaceutical compositions that may be used for the treatment of mature B-cells neoplasms.

### Conditions and Treatments

In certain embodiments, the present invention provides methods for the treatment of mature B-cells neoplasms comprising administering to a subject having or suffering from said pathology any of the compositions described through-out the present specification.

A subject may be diagnosed with a mature B-cells neoplasm following the administration of medical tests well-known to those in the medical profession. In certain embodiments, the mature B-cell neoplasia may be any type of B-cell neoplasia, for example, follicular lymphoma, diffuse large B cell lymphoma (DLBCL), preferably diffuse large B cell lymphoma of the ABC subtype, Burkitt's lymphoma and chronic lymphocytic leukemia (CLL), all derived from germinal centre (GC) B cells. Thus, in a particular embodiment of the present invention, the mature B-cell neoplasia is derived from germinal centre (GC) B cells.

Administration of a composition of the present invention to a subject having a mature B-cell neoplasm may result in one or more clinically desirable outcomes. Such clinically desirable outcomes include the inhibition of tumour cell proliferation and the promotion of tumour cell death in mature B-cells neoplasms. Additional clinically desirable outcomes include the extension of overall survival time of the subject, and/or extension of progression-free survival time of the subject. In certain embodiments, administration of a composition of the invention slows or stops metastatic progression. In certain embodiments, administration of a composition of the invention prevents the recurrence of tumours.

Administration of a composition of the present invention to cancer cells may result in desirable phenotypic effects. In certain embodiments, a composition of the invention may stop, slow or reduce the uncontrolled proliferation of cancer cells. In certain embodiments, a composition of the invention may induce apoptosis in cancer cells. In certain embodiments, a composition of the invention may induce senescence in cancer cells. In certain embodiments, a composition of the invention may reduce cancer cell survival.

### Compositions of the Invention

Provided herein are compositions comprised of at least one lipid for use in delivering miR-28 (including miR-28 carried in vector, in particular in a viral vector such as a lentivirus) or miR-28 mimics, to cells and tissues. In certain embodiments, a lipid is selected to enhance the delivery of the said compound to a particular tissue.

In certain embodiments, a composition comprises at least one lipid. In certain embodiments, a composition comprises at least two lipids. In certain embodiments, a composition comprises at least three lipids. In certain embodiments, a composition comprises at least four lipids.

In certain embodiments, a composition of the invention comprises a cationic lipid, a neutral lipid, a sterol, and a disaggregation lipid.

In certain embodiments, a cationic lipid is 2,2-Dilinoleyl-4-dimethylaminoethyl[1,3]-dioxolane. In certain embodiments, a sterol is cholesterol.

In certain embodiments, a disaggregation lipid is a polyethylene glycol-modified lipid (PEG-modified lipid). In certain embodiments a PEG-modified lipid is PEG-didimyristoyl glycerol (PEG-DMG). In certain embodiments a PEG-modified lipid is PEG-distyryl glycerol (PEG-DSG). In certain embodiments a PEG-modified lipid is PEG-carbamoyl-1,2-dimyristyloxypropylamine (PEG-cDMA).

In certain embodiments, a neutral lipid is a phospholipid. In certain embodiments, a phospholipid is selected from phosphatidylcholine (PC), distearoylphosphatidylcholine (DSPC), and dipalmitoylphosphatidylcholine (DPPC).

In certain embodiments, the composition consists of or consists essentially of a cationic lipid, a neutral lipid, cholesterol, and a PEG-modified lipid. In certain embodiments, a composition consists of or consists essentially of the above lipid mixture in molar ratios of about 20-70% cationic lipid: 5-45% neutral lipid: 20-55% cholesterol: 0.5-15% PEG-modified lipid. In certain embodiments, the composition comprises a cationic lipid, neutral lipid, sterol, and disaggregation lipid in a molar ratio of 50 to 60:7 to 10:30 to 40:1 to 5. In certain embodiments, the molar ratio is 57.5:7.5:31.5:3.5. In certain embodiments, the molar ratio is 60:7.5:31:1.5. In certain embodiments, the molar ratio is 50:10:38.5:1.5.

In certain embodiments, the ratio of total lipid to miR-28 or to the miR-28 mimic is from 5 to 35 (i.e. from 5 to 1 to 35 to 1, lipid weight to oligomeric compound weight). In certain embodiments, the ratio of total lipid to oligomeric compound is from 5 to 15 (i.e. from 5 to 1 to 15 to 1, lipid weight to oligomeric compound weight). In certain embodiments, the ratio of total lipid to oligomeric compound is 5 (i.e. 5 to 1, lipid weight to compound weight). In certain embodiments, the ratio of total lipid to oligomeric compound is 6 (i.e. 6 to 1, lipid weight to compound weight). In certain embodiments, the ratio of total lipid to oligomeric compound is 7 (i.e. 7 to 1, lipid weight to compound weight). In certain embodiments, the ratio of total lipid to oligomeric compound is 8 (i.e. 8 to 1, lipid weight to compound weight). In certain embodiments, the ratio of total lipid to oligomeric compound is 9 (i.e. 9 to 1, lipid weight to compound weight). In certain embodiments, the ratio of total lipid to oligomeric compound is 10 (i.e. 10 to 1, lipid weight to compound weight). In certain embodiments, the ratio of total lipid to oligomeric compound is 11 (i.e. 11 to 1, lipid weight to compound weight).

In certain embodiments, a composition of the invention comprises a miR-28 or a miR-28 mimic, a cationic lipid, a neutral lipid, a sterol, and a disaggregation lipid in a molar ratio of 57.5 to 7.5 to 31.5 to 3.5 wherein the cationic lipid is 2,2-Dilinoleyl-4-dimethylaminoethyl[1,3]-dioxolane (XTC), the neutral lipid is distearoylphosphatidylcholine (DSPC), the sterol is cholesterol and the disaggregation lipid is PEG-didimyristoyl glycerol (PEG-DMG), and wherein the ratio of total lipid to oligomeric compound ratio is 6 to 1 (lipid weight to oligomeric compound weight). In certain embodiments, a composition of the invention comprises a miR-28 or a miR-28 mimic, a cationic lipid, a neutral lipid, a sterol, and a disaggregation lipid in a molar ratio of 57.5 to 7.5 to 31.5 to 3.5 wherein the cationic lipid is 2,2-Dilinoleyl-4-dimethylaminoethyl[1,3]-dioxolane (XTC), the neutral lipid is distearoylphosphatidylcholine (DSPC), the sterol is cholesterol and the disaggregation lipid is PEG-didimyristoyl glycerol (PEG-DMG), and wherein the ratio of total lipid to oligomeric compound ratio is 11 to 1 (lipid weight to oligomeric compound weight).

In certain embodiments, a composition of the invention comprises a miR-28 or a miR-28 mimic, a cationic lipid, a neutral lipid, a sterol, and a disaggregation lipid in a molar ratio of 60 to 7.5 to 31 to 1.5 wherein the cationic lipid is 2,2-Dilinoleyl-4-dimethylaminoethyl[1,3]-dioxolane (XTC), the neutral lipid is distearoylphosphatidylcholine (DSPC), the sterol is cholesterol and the disaggregation lipid is PEG-didimyristoyl glycerol (PEG-DMG), and wherein the ratio of total lipid to the miR-28 or to the miR-28 mimic ratio is 6 to 1 (lipid weight to oligomeric compound weight).

In certain embodiments, a composition of the invention comprises a miR-28 or a miR-28 mimic, a cationic lipid, a neutral lipid, a sterol, and a disaggregation lipid in a molar ratio of 60 to 7.5 to 31 to 1.5 wherein the cationic lipid is 2,2-Dilinoleyl-4-dimethylaminoethyl-[1,3]-dioxo lane (XTC), the neutral lipid is distearoylphosphatidylcholine (DSPC), the sterol is cholesterol and the disaggregation lipid is PEG-didimyristoyl glycerol (PEG-DMG), and wherein the ratio of total lipid to the miR-28 or the miR-28 mimic ratio is 11 to 1 (lipid weight to oligomeric compound weight).

In certain embodiments, a composition of the invention comprises a miR-28 or a miR-28 mimic, a cationic lipid, a neutral lipid, a sterol, and a disaggregation lipid in a molar ratio of 50 to 10 to 38.5 to 1.5 wherein the cationic lipid is 2,2-Dilinoleyl-4-dimethylaminoethyl[1,3]-dioxolane (XTC), the neutral lipid is distearoylphosphatidylcholine (DSPC), the sterol is cholesterol and the disaggregation lipid is PEG-didimyristoyl glycerol (PEG-DMG), and wherein the ratio of total lipid to the miR-28 or the miR-28 mimic ratio is 11 to 1 (lipid weight to oligomeric compound weight).

In certain embodiments, a composition of the invention comprises a miR-28 or a miR-28 mimic, a cationic lipid, a neutral lipid, a sterol, and a disaggregation lipid in a molar ratio of 50 to 10 to 38.5 to 1.5 wherein the cationic lipid is 2,2-Dilinoleyl-4-dimethylaminoethyl[1,3]-dioxolane (XTC), the neutral lipid is distearoylphosphatidylcholine (DSPC), the sterol is cholesterol and the disaggregation lipid is PEG-didimyristoyl glycerol (PEG-DMG), and wherein the ratio of total lipid to the miR-28 or the miR-28 mimic ratio is 10 to 1 (lipid weight to oligomeric compound weight).

In certain embodiments, a composition of the invention comprises a lipid, an aqueous component, and a non-ionic surfactant, wherein the lipid comprises 20-100% by weight of a neutral phospholipid and 0-80% by weight of an oil or wax; the aqueous component comprises a miR-28 or a miR-28 mimic in an aqueous medium; and the surfactant comprises 0.1-50% of the total emulsion by weight. In certain embodiments, the neutral phospholipid is 1,2-dioleoyi-sn-glycero-3-phosphocholine. In certain embodiments, the oil is squalene. In certain embodiments, the surfactant is polysorbate 20. In certain embodiments, the composition comprises an antioxidant. In certain embodiments, the lipid comprises 20-40% phospholipid and 60-80% oil or wax; and the surfactant comprises 40-50% of the total emulsion by weight. Additional lipid-containing compositions are described in US Patent Publication No. 20090306194, which is herein incorporated by reference in its entirety for the description of lipid-containing compositions.

In certain embodiments, it is desirable to target compositions of the invention using targeting moieties that are specific to a cell type or tissue. Targeting of lipid particles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can comprise the entire protein or fragments thereof. Targeting mechanisms generally require that the targeting agents be positioned on the surface of the lipid particle in such a manner that the target moiety is available for interaction with the target, for example, a cell surface receptor. A variety of different targeting agents and methods are known and available in the art, including those described, e.g., in Sapra, P. and Allen, T M, Frog. Lipid Res. 42(5):439-62 (2003); and Abra, R M et al., J. Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

Oligomeric Compounds or miRNA-28 Mimics of the Invention

Provided herein are oligomeric compounds that are designed to mimic miR-28 activity (miR-28 mimics). In certain embodiments, the oligomeric compounds comprise oligonucleotides having nucleobase identity to the nucleobase sequence of miR-28, are thus designed to mimic miR-28 activity. In certain embodiments, the oligomeric compound comprises an oligonucleotide hybridized to a complementary strand.

Compositions of the present invention comprise oligomeric compounds comprising oligonucleotides having nucleobase sequences that share identity with endogenous miRNA-28 or miRNA-28 precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with miRNA-28 precursor may be up to 100 linked nucleosides in length.

In certain embodiments, an oligonucleotide has a nucleobase sequence at least 60%, 65%, 70%, 75%, 80%, 85%, 90%, 95%, 97%, 98% or 99% identical to the miRNA-28 over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA-28. In certain embodiments, the miR-28 has the nucleobase sequence of SEQ ID NO: 1.

Compositions of the present invention may comprise oligonucleotides having a percentage region identity and percentage overall identity that are different from one another. In certain embodiments, a region of the nucleobase sequence of an oligonucleotide is 100% identical to the nucleobase sequence of the miRNA-28, but the oligonucleotide does not have 100% overall identity to the entire miRNA-28.

Compositions of the present invention may comprise oligonucleotides having seed region identity with a miRNA-28. In certain embodiments, the nucleobase sequence of an oligonucleotide has at least 80% seed region identity with the nucleobase sequence of a miRNA-28. In certain embodiments, the nucleobase sequence of an oligonucleotide has at least 85% seed region identity with the nucleobase sequence of a miRNA-28. In certain embodiments, the nucleobase sequence of an oligonucleotide has at least 90% seed region identity with the nucleobase sequence of a miRNA-28. In certain embodiments, the nucleobase sequence of an oligonucleotide has at least 95% seed region identity with the nucleobase sequence of a miRNA-28. In certain embodiments, the nucleobase sequence of an oligonucleotide has 100% seed region identity with the nucleobase sequence of a miRNA-28.

The seed region of a miRNA-28 may comprise one of several sequences, thus seed region identity may be calculated differently depending on the selection of a particular seed sequence. In certain embodiments, the nucleobase sequence of an oligonucleotide has at least 80% seed region identity with the nucleobase sequence of a miRNA-28, and at least 80% overall identity with the miRNA-28. In certain embodiments, the nucleobase sequence of an oligonucleotide has at least 80% seed region identity with the nucleobase sequence of a miRNA, and at least 85% overall identity with the miRNA-28. In certain embodiments, the nucleobase sequence of an oligonucleotide has at least 80% seed region identity with the nucleobase sequence of a miRNA, and at least 90% overall identity with the miRNA-28. In certain embodiments, the nucleobase sequence of an oligonucleotide has at least 80% seed region identity with the nucleobase sequence of a miRNA-28, and at least 95% overall identity with the miRNA-28. In certain embodiments, the nucleobase sequence of an oligonucleotide has at least 80% seed region identity with the nucleobase sequence of a miRNA-28, and 100% overall identity with the miRNA-28.

In certain embodiments, the nucleobase sequence of an oligonucleotide has at least 85% seed region identity with the nucleobase sequence of a miRNA-28, and at least 80% overall identity with the miRNA-28. In certain embodiments, the nucleobase sequence of an oligonucleotide has at least 85% seed region identity with the nucleobase sequence of a miRNA-28, and at least 85% overall identity with the miRNA-28. In certain embodiments, the nucleobase sequence of an oligonucleotide has at least 85% seed region identity with the nucleobase sequence of a miRNA-28, and at least 90% overall identity with the miRNA-28. In certain embodiments, the nucleobase sequence of an oligonucleotide has at least 85% seed region identity with the nucleobase sequence of a miRNA-28, and at least 95% overall identity with the miRNA-28. In certain embodiments, the nucleobase sequence of an oligonucleotide has at least 85% seed region identity with the nucleobase sequence of a miRNA-28, and 100% overall identity with the miRNA-28.

In certain embodiments, the nucleobase sequence of an oligonucleotide has at least 90% seed region identity with the nucleobase sequence of a miRNA-28, and at least 80% overall identity with the miRNA-28. In certain embodiments, the nucleobase sequence of an oligonucleotide has at least 90% seed region identity with the nucleobase sequence of a miRNA-28, and at least 85% overall identity with the miRNA-28. In certain embodiments, the nucleobase sequence of an oligonucleotide has at least 90% seed region identity with the nucleobase sequence of a miRNA-28, and at least 90% overall identity with the miRNA-28. In certain embodiments, the nucleobase sequence of an oligonucleotide has at least 90% seed region identity with the nucleobase sequence of a miRNA-28, and at least 95% overall identity with the miRNA-28. In certain embodiments, the nucleobase sequence of an oligonucleotide has at least 90% seed region identity with the nucleobase sequence of a miRNA-28, and 100% overall identity with the miRNA-28.

In certain embodiments, the nucleobase sequence of an oligonucleotide has at least 95% seed region identity with the nucleobase sequence of a miRNA-28, and at least 80% overall identity with the miRNA-28. In certain embodiments, the nucleobase sequence of an oligonucleotide has at least 95% seed region identity with the nucleobase sequence of a miRNA-28, and at least 85% overall identity with the miRNA-28. In certain embodiments, the nucleobase sequence of an oligonucleotide has at least 95% seed region identity with the nucleobase sequence of a miRNA-28, and at least 90% overall identity with the miRNA-28. In certain embodiments, the nucleobase sequence of an oligonucleotide has at least 95% seed region identity with the nucleobase sequence of a miRNA-28, and at least 95% overall identity with the miRNA-28. In certain embodiments, the nucleobase sequence of an oligonucleotide has at least 95% seed region identity with the nucleobase sequence of a miRNA-28, and 100% overall identity with the miRNA-28.

In certain embodiments, the nucleobase sequence of an oligonucleotide has 100% seed region identity with the nucleobase sequence of a miRNA-28, and at least 80% overall identity with the miRNA-28. In certain embodiments, the nucleobase sequence of an oligonucleotide has 100% seed region identity with the nucleobase sequence of a miRNA-28, and at least 85% overall identity with the miRNA-28. In certain embodiments, the nucleobase sequence of an oligonucleotide has 100% seed region identity with the nucleobase sequence of a miRNA, and at least 90% overall identity with the miRNA-28. In certain embodiments, the nucleobase sequence of an oligonucleotide has 100% seed region identity with the nucleobase sequence of a miRNA-28, and at least 95% overall identity with the miRNA-28. In certain embodiments, the nucleobase sequence of an oligonucleotide has 100% seed region identity with the nucleobase sequence of a miRNA-28, and 100% overall identity with the miRNA-28.

In certain embodiments, an oligonucleotide has a nucleobase sequence having one non-identical nucleobase with respect to the nucleobase sequence of a mature miRNA-28, or a precursor thereof. In certain embodiments, an oligonucleotide has a nucleobase sequence having two non-identical nucleobases with respect to the nucleobase sequence of a miRNA-28, or a precursor thereof. In certain such embodiments, an oligonucleotide has a nucleobase sequence having no more than two non-identical nucleobases with respect to the nucleobase sequence of a mature miRNA-28, or a precursor thereof. In certain such embodiments, the non-identical nucleobases are contiguous. In certain such embodiments, the non-identical nucleobases are not contiguous.

In certain embodiments, an oligomeric compound for use in a composition described herein comprises an oligonucleotide hybridized to a complementary oligonucleotide, i.e. the oligomeric compound is a double-stranded oligomeric compound.

A double-stranded oligomeric compound may be from 7 to 30 basepairs in length. In certain embodiments, a double-stranded oligomeric compound is from 15 to 30 basepairs in length. In certain embodiments, a double-stranded oligomeric compound is from 19 to 23 basepairs in length. In certain embodiments, a double-stranded oligomeric compound is 19 basepairs in length. In certain embodiments, a double-stranded oligomeric compound is 20 basepairs in length. In certain embodiments, a double-stranded oligomeric compound is 21 basepairs in length. In certain embodiments, a double-stranded oligomeric compound is 22 basepairs in length. In certain embodiments, a double-stranded oligomeric compound is 23 basepairs in length. In certain embodiments, the hybridization of an oligonucleotide to a complementary oligonucleotide forms at least one blunt end. In certain such embodiments, the hybridization of an oligonucleotide to a complementary oligonucleotide forms a blunt end at each terminus of the double-stranded oligomeric compound.

The hybridization of an oligonucleotide to a complementary oligonucleotide may result in the formation of one or more overhangs, where one or more additional nucleosides of at least one terminus of the oligonucleotide do not have a corresponding nucleobase in the complementary oligonucleotide with which to pair through hydrogen bonding. In such cases, the hybridization of the oligonucleotide to the complementary oligonucleotide results in the formation of a central complementary region. The central complementary region can tolerate mismatches, provided that there is sufficient complementarity to permit hybridization. In certain embodiments, there are 0, 1, 2, or 3 mismatches in the central complementary region.

In certain embodiments, a terminus of an oligonucleotide comprises one or more additional linked nucleosides relative to the number of linked nucleosides of the complementary oligonucleotide. In certain embodiments, the one or more additional nucleosides are at the 5′ terminus of an oligonucleotide. In certain embodiments, the one or more additional nucleosides are at the 3′ terminus of an oligonucleotide. In certain embodiments, at least one nucleobase of a nucleoside of the one or more additional nucleosides is complementary to the target RNA. In certain embodiments, each nucleobase of each one or more additional nucleosides is complementary to the target RNA. In certain embodiments, a terminus of the complementary oligonucleotide comprises one or more additional linked nucleosides relative to the number of linked nucleosides of an oligonucleotide. In certain embodiments, the one or more additional linked nucleosides are at the 3′ terminus of the complementary oligonucleotide. In certain embodiments, the one or more additional linked nucleosides are at the 5′ terminus of the complementary oligonucleotide. In certain embodiments, two additional linked nucleosides are linked to a terminus. In certain embodiments, one additional nucleoside is linked to a terminus. In certain embodiments, a composition of the present invention comprises an oligomeric compound comprising an oligonucleotide having nucleobase identity to miR-28 and a complementary oligonucleotide.

In certain embodiments, the oligomeric compound comprises an oligonucleotide conjugated to one or more moieties which enhance the activity, cellular distribution or cellular uptake of the resulting antisense oligonucleotides. In certain such embodiments, the moiety is a cholesterol moiety or a lipid moiety. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to an oligonucleotide. In certain embodiments, a conjugate group is attached to an oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimido methyl) cyclo hex ane-1-carboxylate (SMCC), 6-amino hexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the oligomeric compound comprises an oligonucleotide having one or more stabilizing groups that are attached to one or both termini of the oligonucleotide to enhance properties such as, for example, nuclease stability. Included in stabilizing groups are cap structures. These terminal modifications protect an oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-T-inverted nucleotide moiety, a 3′-T-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

The nucleobase sequences set forth herein, including but not limited to those found in the Examples and in the sequence listing, are independent of any modification to the nucleic acid.

Although the sequence listing accompanying this filing identifies each nucleobase sequence as either “RNA” or “DNA” as required, in reality, those sequences may be modified with any combination of chemical modifications. One of skill in the art will readily appreciate that such designation as “RNA” or “DNA” to describe modified oligonucleotides is somewhat arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases.

Modifications to the Oligomeric Compounds or miRNA-28 Mimics of the Invention.

Oligonucleotides of the present invention may comprise one or more modifications to a nucleobase, sugar, and/or internucleoside linkage. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In certain embodiments, an oligonucleotide of the present invention comprises one or more modified nucleosides. In certain such embodiments, a modified nucleoside is a stabilizing nucleoside. An example of a stabilizing nucleoside is a sugar-modified nucleoside.

In certain embodiments, a modified nucleoside is a sugar-modified nucleoside. In certain such embodiments, the sugar-modified nucleosides can further comprise a natural or modified heterocyclic base moiety and/or a natural or modified internucleoside linkage and may include further modifications independent from the sugar modification. In certain embodiments, a sugar modified nucleoside is a 2′-modified nucleoside, wherein the sugar ring is modified at the 2′ carbon from natural ribose or 2′-deoxy-ribose.

In certain embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

In certain embodiments, the bicyclic sugar moiety comprises a bridge group between the T and the 4′-carbon atoms. In certain such embodiments, the bridge group comprises from 1 to 8 linked biradical groups. In certain embodiments, the bicyclic sugar moiety comprises from 1 to 4 linked biradical groups. In certain embodiments, the bicyclic sugar moiety comprises 2 or 3 linked biradical groups. In certain embodiments, the bicyclic sugar moiety comprises 2 linked biradical groups. In certain embodiments, a linked biradical group is selected from —O—, —S—, —N(R1)-, —C(R1)(R2)-, —C(R1)═C(R1)-, —C(R1)═N—, —C(═NR1)-, —Si(R1)(R2)-, —S(═O)2-, —S(═O)—, —C(═O)— and —C(═S)—; where each R1 and R2 is, independently, H, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, a heterocycle radical, a substituted hetero-cycle radical, heteroaryl, substituted heteroaryl, C5-C7 alicyclic radical, substituted C5-C7 alicyclic radical, halogen, substituted oxy (—O—), amino, substituted amino, azido, carboxyl, substituted carboxyl, acyl, substituted acyl, CN, thiol, substituted thiol, sulfonyl (S(═O)2-H), substituted sulfonyl, sulfoxyl (S(═O)—H) or substituted sulfoxyl; and each substituent group is, independently, halogen, C1—C12 alkyl, substituted C1—C12 alkyl, C2—C12 alkenyl, substituted C2—C12 alkenyl, C2—C12 alkynyl, substituted C2—C12 alkynyl, amino, substituted amino, acyl, substituted acyl, C1—C12 aminoalkyl, C1—C12 aminoalkoxy, substituted C1—C12 aminoalkyl, substituted C1—C12 aminoalkoxy or a protecting group.

In some embodiments, the bicyclic sugar moiety is bridged between the 2′ and 4′ carbon atoms with a biradical group selected from —O—(CH2)p-, —O—CH2—, —O—CH2CH2—, —O—CH(alkyl)-, —NH—(CH2)p-, —N(alkyl)-(CH2)p-, —O—CH(alkyl)-, —(CH(alkyl))—(CH2)p-, —NH—O—(CH2)p-, —N(alkyl)—O—(CH2)p-, or —O—N(alkyl)-(CH2)p-, wherein p is 1, 2, 3, 4 or 5 and each alkyl group can be further substituted. In certain embodiments, p is 1, 2 or 3.

In certain embodiments, a 2′-modified nucleoside comprises a T-substituent group selected from halo, allyl, amino, azido, SH, CN, OCN, CF3, OCF3, O—, S—, or N(Rm)-alkyl; O—, S—, or N(Rm)-alkenyl; O—, S— or N(Rm)-alkynyl; O-alkylenyl-O-alkyl, alkynyl, alkaryl, aralkyl, O-alkaryl, O-aralkyl, O(CH2)2SCH3, O—(CH2)2-O—N(Rm)(Rn) or O—CH2-C(═O)—N(Rm)(Rn), where each Rm and Rn is, independently, H, an amino protecting group or substituted or unsubstituted C1—C10 alkyl. These T-substituent groups can be further substituted with one or more substituent groups independently selected from hydroxyl, amino, alkoxy, carboxy, benzyl, phenyl, nitro (NO2), thiol, thioalkoxy (S-alkyl), halogen, alkyl, aryl, alkenyl and alkynyl.

In certain embodiments, a 2′-modified nucleoside comprises a 2′-substituent group selected from F, NH2, N3, OCF3, O—CH3, O(CH2)3NH2, CH2—CH═CH2, O—CH2-CH═CH2, OCH2CH2OCH3, O(CH2)2SCH3, O—(CH2)2-O—N(Rm)(Rn), O(CH2)2O(CH2)2N(CH3)2, and N-substituted acetamide (O—CH2-C(═O)—N(Rm)(Rn) where each Rm and Rn is, independently, H, an amino protecting group or substituted or unsubstituted C1—C10 alkyl.

In certain embodiments, a 2′-modified nucleoside comprises a 2′-substituent group selected from F, OCF3, O—CH3, OCH2CH2OCH3, 2′-O(CH2)2SCH3, O—(CH2)2-O—N(CH3)2, —O(CH2)2O(CH2)2N(CH3)2, and O—CH2-C(═O)—N(H)CH3.

In certain embodiments, a 2′-modified nucleoside comprises a 2′-substituent group selected from F, O—CH3, and OCH2CH2OCH3.

In certain embodiments, a sugar-modified nucleoside is a 4′-thio modified nucleoside. In certain embodiments, a sugar-modified nucleoside is a 4′-thio-2′-modified nucleoside. A 4′-thio modified nucleoside has a β-D-ribonucleoside where the 4′-O replaced with 4′-S. A 4′-thio-T-modified nucleoside is a 4′-thio modified nucleoside having the 2′-OH replaced with a 2′-substituent group. Suitable 2′-substituent groups include T-OCH3, 2′-O—(CH2)2-OCH3, and T-F.

In certain embodiments, an oligonucleotide of the present invention comprises one or more internucleoside modifications. In certain such embodiments, each internucleoside linkage of a modified oligonucleotide is a modified internucleoside linkage. In certain embodiments, a modified internucleoside linkage comprises a phosphorus atom.

In certain embodiments, an oligonucleotide of the present invention comprises at least one phosphorothioate internucleoside linkage. In certain embodiments, each internucleoside linkage of a modified oligonucleotide is a phosphorothioate internucleoside linkage.

In certain embodiments, a modified internucleoside linkage does not comprise a phosphorus atom. In certain such embodiments, an internucleoside linkage is formed by a short chain alkyl internucleoside linkage. In certain such embodiments, an internucleoside linkage is formed by a cycloalkyl internucleoside linkages. In certain such embodiments, an internucleoside linkage is formed by a mixed heteroatom and alkyl internucleoside linkage. In certain such embodiments, an internucleoside linkage is formed by a mixed heteroatom and cycloalkyl internucleoside linkages. In certain such embodiments, an internucleoside linkage is formed by one or more short chain heteroatomic internucleoside linkages. In certain such embodiments, an internucleoside linkage is formed by one or more heterocyclic internucleoside linkages. In certain such embodiments, an internucleoside linkage has an amide backbone. In certain such embodiments, an internucleoside linkage has mixed N, O, S and CH2 component parts.

In certain embodiments, an oligonucleotide comprises one or more modified nucleobases. In certain embodiments, a modified oligonucleotide comprises one or more 5-methylcytosines. In certain embodiments, each cytosine of a modified oligonucleotide comprises a 5-methylcytosine.

In certain embodiments, a modified nucleobase is selected from 5-hydroxymethyl cytosine, 7-deazaguanine and 7-deazaadenine. In certain embodiments, a modified nucleobase is selected from 7-deaza-adenine, 7-deazaguanosine, 2-aminopyridine and 2-pyridone. In certain embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In certain embodiments, a modified nucleobase comprises a polycyclic heterocycle. In certain embodiments, a modified nucleobase comprises a tricyclic heterocycle. In certain embodiments, a modified nucleobase comprises a phenoxazine derivative. In certain embodiments, the phenoxazine can be further modified to form a nucleobase known in the art as a G-clamp.

Compositions Comprising miRNA-28 Expression Vectors

Expression vectors, such as lentiviral vectors, that contain a miRNA-28 sequence, or a precursor thereof, are also useful in the methods described herein, for the delivery of an miRNA-28 or precursor thereof to a cell or tissue (see examples). Thus provided herein are expression vectors that comprise a miRNA-28 sequence, preferably SEQ ID NO 2, or a precursor thereof, optionally associated with a regulatory element that directs the expression of the miRNA-28 sequence or precursor thereof. The choice of vector and/or expression control sequences to which the miRNA-28 sequence, or precursor thereof, is operably linked depends on the functional properties desired, and the cell type to which the vector is to be delivered. In certain embodiments, the expression vector is a retroviral vector. In certain embodiments, the expression vector is an adenoviral vector. In certain embodiments, the expression vector is an adeno-associated viral vector. In certain embodiments, the expression vector is a lentiviral vector, in particular by lentiviral construct of example 1.

### Pharmaceutical Compositions of the Invention

In certain embodiments, any of the compositions described herein can be prepared as a pharmaceutical composition for the treatment of a mature B-cell neoplasms. Suitable administration routes include, but are not limited to, oral, rectal, transmucosal, intestinal, enteral, topical, suppository, through inhalation, intrathecal, intraventricular, intraperitoneal, intranasal, intraocular, intratumoral, and parenteral (e.g., intravenous, intramuscular, intramedullary, and subcutaneous). An additional suitable administration route includes chemoembolization. In certain embodiments, pharmaceutical intrathecals are administered to achieve local rather than systemic exposures. For example, pharmaceutical compositions may be injected directly in the area of desired effect (e.g., into a tumour).

In certain embodiments, a pharmaceutical composition of the present invention is administered in the form of a dosage unit (e.g., tablet, capsule, bolus, etc.). In certain embodiments, such pharmaceutical compositions comprise a miRNA-28 mimic in a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, a pharmaceutical composition of the present invention comprises a dose of a miRNA-28 mimic selected from 25 mg, 50 mg, 75 mg, 100 mg, 150 mg, 200 mg, 250 mg, 300 mg, 350 mg, 400 mg, 500 mg, 600 mg, 700 mg, and 800 mg.

In certain embodiments, a pharmaceutical agent is a sterile lyophilized miRNA-28 mimic that is reconstituted with a suitable diluent, e.g., sterile water for injection or sterile saline for injection. The reconstituted product is administered as a subcutaneous injection or as an intravenous infusion after dilution into saline. The lyophilized drug product consists of a miRNA-28 mimic which has been prepared in water for injection, or in saline for injection, adjusted to pH 7.0-9.0 with acid or base during preparation, and then lyophilized. The lyophilized drug product may be packaged in a 2 mL Type I, clear glass vial (ammonium sulfate-treated), stoppered with a bromobutyl rubber closure and sealed with an aluminum FLIP-OFF® overseal.

In certain embodiments, the compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

In certain embodiments, pharmaceutical compositions of the present invention comprise one or more miRNA-28 mimics and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition of the present invention is prepared using known techniques, including, but not limited to mixing, dissolving, granulating, dragee-making, levigating, emulsifying, encapsulating, entrapping or tabletting processes.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

In certain embodiments, a pharmaceutical composition of the present invention is a solid (e.g., a powder, tablet, and/or capsule). In certain of such embodiments, a solid pharmaceutical composition comprising one or more miRNA-28 mimics is prepared using ingredients known in the art, including, but not limited to, starches, sugars, diluents, granulating agents, lubricants, binders, and disintegrating agents.

In certain embodiments, a pharmaceutical composition of the present invention is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition of the present invention comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition of the present invention comprises a co-solvent system. Certain of such co-solvent systems comprise, for example, benzyl alcohol, a nonpolar surfactant, a water-miscible organic polymer, and an aqueous phase.

In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

In certain embodiments, a pharmaceutical composition of the present invention is prepared for oral administration. In certain of such embodiments, a pharmaceutical composition is formulated by combining one or more compounds comprising an miRNA-28 mimic with one or more pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. In certain embodiments, pharmaceutical compositions for oral use are obtained by mixing miRNA-28 mimics and one or more solid excipients. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, pharmaceutical compositions for oral administration are soft, sealed capsules made of gelatin and a plasticizer, such as glycerol or sorbitol. In certain soft capsules, one or more pharmaceutical agents of the present invention are be dissolved or suspended in suitable liquids, such as fatty oils, liquid paraffin, or liquid polyethylene glycols. In addition, stabilizers may be added.

In certain embodiments, pharmaceutical compositions are prepared for buccal administration. Certain of such pharmaceutical compositions are tablets or lozenges formulated in conventional manner.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions. In certain embodiments, a pharmaceutical composition is prepared for transmucosal administration. In certain of such embodiments penetrants appropriate to the barrier to be permeated are used in the formulation. Such penetrants are generally known in the art. In certain embodiments, a pharmaceutical composition is prepared for administration by inhalation. Certain of such pharmaceutical compositions for inhalation are prepared in the form of an aerosol spray in a pressurized pack or a nebulizer. Certain of such pharmaceutical compositions comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a pharmaceutical agent of the invention and a suitable powder base such as lactose or starch.

In certain embodiments, a pharmaceutical composition is prepared for rectal administration, such as a suppositories or retention enema. Certain of such pharmaceutical compositions comprise known ingredients, such as cocoa butter and/or other glycerides.

In certain embodiments, a pharmaceutical composition is prepared for topical administration. Certain of such pharmaceutical compositions comprise bland moisturizing bases, such as ointments or creams. Exemplary suitable ointment bases include, but are not limited to, petrolatum, petrolatum plus volatile silicones, and lanolin and water in oil emulsions. Exemplary suitable cream bases include, but are not limited to, cold cream and hydrophilic ointment.

In certain embodiments, a pharmaceutical composition of the present invention comprises an miRNA-28 mimic or a composition comprising a miRNA-28 expression vector in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art.

In certain embodiments, any of the compositions of the present invention is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

### Kits of the Invention

The present invention also provides kits. In some embodiments, the kits comprise one or more compounds comprising an oligonucleotide of 7 to 30 linked nucleosides, wherein the nucleobase sequence of the oligonucleotide has identity to miR-28. The compounds can be any of the compounds described herein, and can have any of the modifications described herein. In some embodiments, the compounds can be present within a vial. A plurality of vials, such as 10, can be present in, for example, dispensing packs. In some embodiments, the vial is manufactured so as to be accessible with a syringe.

In some embodiments, the kits may be used for administration of the compound to a subject. In such instances, in addition to compounds having identity to miR-28, the kit can further comprise one or more of the following: syringe, alcohol swab, cotton ball, and/or gauze pad. In some embodiments, the compounds having identity to miR-28 can be present in a pre-filled syringe (such as a single-dose syringes with, for example, a 27 gauge, ½ inch needle with a needle guard), rather than in a vial. A plurality of pre-filled syringe can be present in, for example, dispensing packs. The kit can also contain instructions for administering the compounds having identity to miR-28.

The foregoing description of the specific embodiments so fully reveals the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

The following examples are presented in order to more fully illustrate some embodiments of the invention. They should, in no way be construed, however, as limiting the broad scope of the invention.

### EXAMPLES

### Example 1

### Materials and Methods

miR-28 detection by quantitative reverse transcriptase-polymerase chain reaction RNA was extracted with Trizol (Invitrogen). For detection of mouse and human mature miR-28-5p by qRT-PCR, miR-28 miRCURY LNA primers (Exiqon) were used. U6 amplification was used as normalization control. The amplification was performed in a 7900HT fast real-time PCR system (Applied Biosystems).

### Cell Culture

Mouse primary B cells were purified from spleens of 6-8 weeks C57/BL6 mice by anti-CD43 immunomagnetic depletion (Miltenyi Biotech) and cultured in RPMI medium containing 10% fetal calf serum (FCS), 25 μg/ml LPS (Sigma), 10 ng/ml IL-4 (Peprotech), 10mM Hepes (Gibco), and 50 μM β-mercaptoethanol (Gibco). The NIH-293T and 3T3 cell lines were cultured in DMEM containing 10% FCS. The human B cell lymphoma cell lines (Ramos, Raji and MD901) and the Jurkat T cell line were cultured in RPMI medium containing 10% FCS and 10 mM Hepes (Gibco).

### Cellular Assays

Apoptosis was measured using Annexin V eFluor450 kit (eBioscience) and anti-active Caspase-3 staining (Clone C92-605) from BD Pharmingen. Proliferation was assessed with Cell Trace Violet (Invitrogen) and analysis was performed using the FlowJo proliferation platform. Cell cycle analysis was performed with propidium iodide staining. Replication was assessed using FITC BrdU Flow Kit (BD Pharmingen).

### Expression Constructs and Transductions

### Retroviral Constructs

For retroviral overexpression of miR-28, the DNA fragment containing the precursor sequence of miR-28 and its flanking 50-bp-long genomic context were cloned in XhoI-EcoRI sites of the pre-miRNA GFP vector, as previously described (de Yebénes 2008). The empty p-miRNA Control vector was generated by liberating the insert, generating blunt ends with T4 polymerase and religating with T4 ligase. For retroviral inhibition of the endogenous miRNA, miR-28 sponges were generated as described in de Yebenés 2014. Briefly, 4 complementary sequences to the mature miR-28 5p were cloned in tandem within the 3′UTR of GFP into the MGP vector by annealing and subsequent extension with Klenow DNA polymerase (New England Biolabs) of two partially complementary oligonucleotides:

(forward)
5′tcgactagctagaacgcggccgcctcaatagactgtgagctccttcga
tctcaatagactgtgagetccttaccggtctcaat-3′
(reverse)
5′-gaagtcagtccgattctcgagaaggagctcacagtctattgaggtga
aaggagctcacagtctattgagaccggtaaggagctc-3′.

Retroviral supernatants were produced by transient calcium phosphate transfection of NIH-293T cells with pCL-ECO (Imgenex) and p-miR-28 and/or miR-28 sponge retroviral vectors. Mouse splenic B cells and total bone marrows were transduced with retroviral supernatants for 16-20 h in the presence of 8 μg/ml polybrene (Sigma-Aldrich).

### Lentiviral Constructs

For inducible expression of miR-28, its precursor sequence was cloned into the pTRIPZ vector (Thermo scientific) in a Tet-on configuration. The pre-miRNA sequence was replaced by a scrambled sequence to obtain the pTRIPZ Scramble Control. Lentiviral supernatants were obtained by transient calcium phosphate transfection of NIH-293T cells with VSVG, Δ9.8 and pTRIPZ. Human lymphoma cell lines were transduced with lentiviral supernatants for 16 h in the presence of 8 μg/ml polybrene. 48 h later, selection of the infected cells was achieved by adding 0.4 ug/mL puromycin to the culture medium for 3 days. For the induction of miR-28 or the Scramble sequence 0.5 ug/ml doxycycline was added and RFP+ was measured in a FacsCalibur cytometer.

### Mice

Lambda-Myc Transgenic mice (Kovalchuck et al J Ex Med 2000) were kindley provided by Dr. Miguel Campanero. NOD/SCID/IL-2rgnull (NSG) mice, C57BL/6 (Harlan) mice and CD45.1 C57BL/6 were bred in house under pathogen-free conditions. All animal procedures conformed to European Union Directive 2010/63EU and Recommendation 2007/526/EC regarding the protection of animals used for experimental and other scientific purposes, enforced in Spanish law under Real Decreto 1201/2005.

### Bone Marrow Reconstitution

A total of 5×106 total bone marrow cells from CD45.1 C57BL/6, donor mice were injected intravenously into 8- to 12-week-old CD45.2 C57BL/6 mice that had been lethally irradiated (2×550 cGy) 24 hr before the reconstitution. Neomicin (2 mg/ml) was added in the drinking water of the mice until complete reconstitution.

### Immunizations (T Cell Dependent Immunizations)

Groups of 6-11 littermate mice were immunized by footpad injection with 50 μg of NP-CGG (Biosearch Technologies) in complete Freund's adjuvant. Mice were re-immunized (secondary immunizations) with 50 μg of NP-CGG in incomplete Freund's adjuvant. Mice injected with PBS were used as non-immunized controls. For immunizations with sheep red blood cells (SRBC), 100×10⁶ cells were intravenously injected in the tail vain and mice were sacrificed between 3-14 days later, as indicated in the corresponding experiment.

### Isolation and Analysis of Ex-Vivo B Cell Subsets by FACS.

For the miR-28 kinetics during GC reaction experiment, naïve (CD19+Fas-GL7-) and GC B cells (CD19+Fas+GL7+) were purified by flow-activated cell sorting from spleens at different time points after immunization with sheep red blood cells (SRBC): After initiation of the GC response at 3-4 days post immunization, after 5-6 days (for early GC samples) or after 10 and 14 days (for late GC samples). Dark zone and light zone GC B cells were separated between 9 to 11 days after immunization by staining the GC B cells with biotinilated anti-CXCR4 (don 2B11 from eBiosciences) and anti-CD86 (BD Pahrmingen). GC B cells were also obtained from Peyer's patches by cell sorting with a FACS Aria cell sorter (BD Biosciences). Analysis of lymphoid populations from bone marrow, spleen and Peyer's patches were performed by preparing suspensions of live cells (7AAD or DAPI negative) and incubating the cells with unlabeled anti-mouse CD16/CD32 antibodies (BD Pharmingen) to block Fc receptors. The cells were then labeled with the following antibodies: anti-CD19-APC, anti-CD19-biotin, anti-CD3-APC, anti-CD45.2-PerCpCy5, anti-CD23-biotin, anti-CD138-PE, anti-IgD-biotin, anti-IgG1-biotin, anti-IgM-biotin, anti-IgA-biotin, PE-Cy7-streptavidin, anti-CD21-PE (all from BD

Pharmingen); anti-B220-vioblue (Miltenyi Biotech); anti-GL7-FITC, anti-GL7-Alexa647 and anti-CD93-APC (BD eBioscience); and anti-IgM-PE (Invitrogen). Labeled cells were analyzed on a FACSCanto flow cytometer (BD Biosciences) with Facsdiva software.

### Subcutaneous Lymphoma Xenograft Model

NSG mice (6-8 weeks) received subcutaneous flank injections of lymphoma cells (2×10⁶ of non-transduced Ramos cells or 1×10⁷ cells in the case of the lentiviraly transduced human tumoral cells and of mouse primary Lambda-Myc lymphoma cells) re-suspended in 100 μL PBS and mixed with 100 μl Matrigel (BD Biosciences) at 1:1 proportions. When the transduced cells were used, doxycycline (Sigma-Aldritch) was administered to the animals one week before the xenograft injection (tumor development experiment) or once the tumors reached 200 mm³ (tumor regression experiments) at a concentration of 0.04% through their drinking water. To establish subcutaneous Lambda-Myc primary tumors, first enlarged lymph nodes and/or spleens were extracted from 2-to-4 months-old Lambda-Myc sick animals and single cell suspensions were prepared in 2% FCS PBS using a cell strainer. Tumor growth was measured 3 times per week with a digital caliper using the following formula: volume=(width)2×length/2. Animals were killed when the tumor reached a volume greater than 2000 mm3 and all tumors were extracted for further FACS and histopathological analysis.

### Systemic Lymphoma Xenograft Model

2×10⁶ of Lambda-Myc primary Burkitt lymphoma cells were intravenously injected in the tail vain of receptor NSG mice. Tumor burden was assessed by periodical bleeding.

miR-28 Replacement and Therapeutic Regimen

MiR-28 mimic (Ambion) was used both in vitro and in vivo to replace endogenous miR-28. For in vitro treatment of primary Lambda-Myc tumors, the tumor cells were transfected with 10 μM of mimic or negative control (Ambion), using Lipofectamine RNAiMAX reagent and OptiMEM medium (both from Life technologies) and following the manufacturer instructions. For in vivo treatment, 3 mg/ml of HPLC purified miR-28 mimic (Ambion) was mixed at 1:1 proportions in complexation buffer (Ambion) and afterwords the resulting solution with Invivofectamin (Ambion) in 1:1 proportions. The solution was incubated at 50° C. 30 minutes and dyalized. For intratumoral treatment final concentrations of 0.1 nM and 0.5 nM were used, injecting the mimic 3 times after tumor establishment separated by 3-4 days. Intravenous treatment was achieved by injecting in the tail vein two doses of 5 mg of mimic per mouse kg (5 mg/kg), separated by 4 days.

### Histopathology and Toxicity Studies

Organs were fixed in 10% buffered formalin and embedded in paraffin. Sections were stained with hematoxylin (Sigma), anti-Pax5 (Santa Cruz Biotechnology), anti-CD3ϵ (Santa Cruz Biotechnology), anti-ki-67 (Abcam), or anti Caspase-3. Quantifications were performed using Image-J. Tissue damage (toxicity studies) and neoplasms where classified by a pathologist according to their histopathological and immunophenotypic profile following the criteria of the consensus international system. In addition to the toxicity studies in the xenografted NSG mice, we conducted specific toxicity studies in C57/BL6 mice. Mice (n=8) were randomized in three groups. Mice were weighed and examined every other day. Mice were euthanized at indicated times when blood and tissues were harvested and microscopically examined.

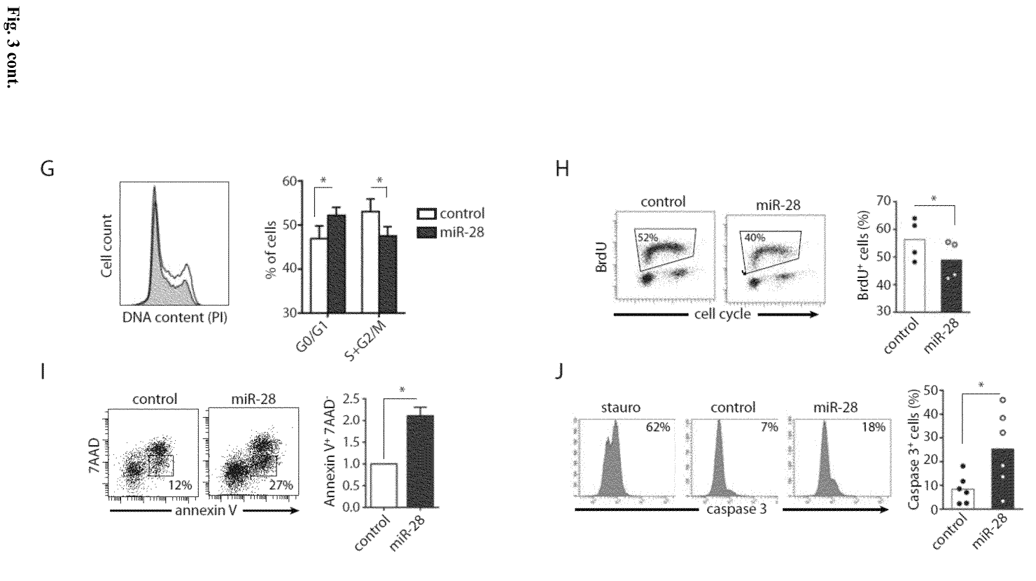

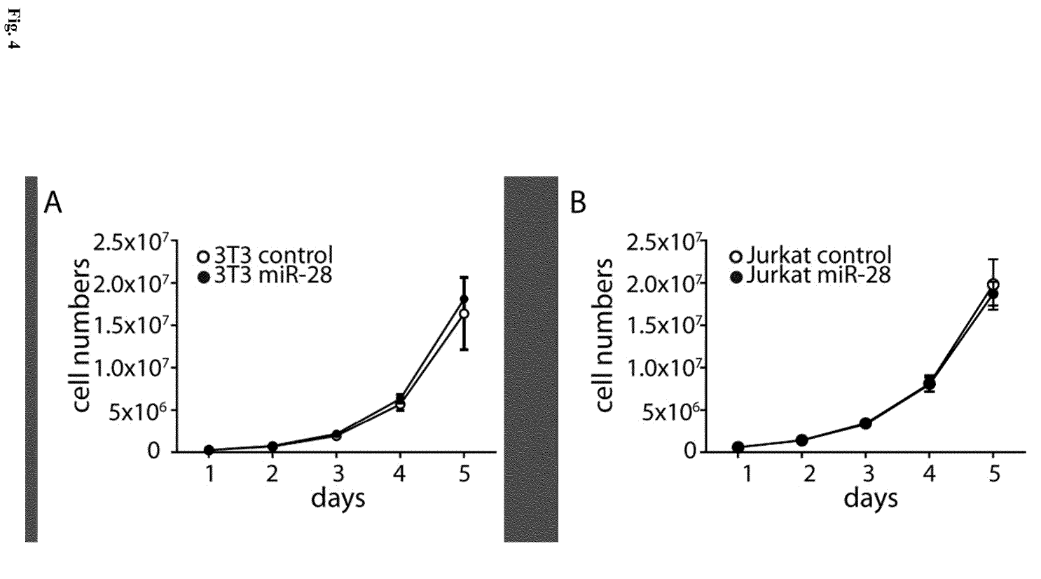

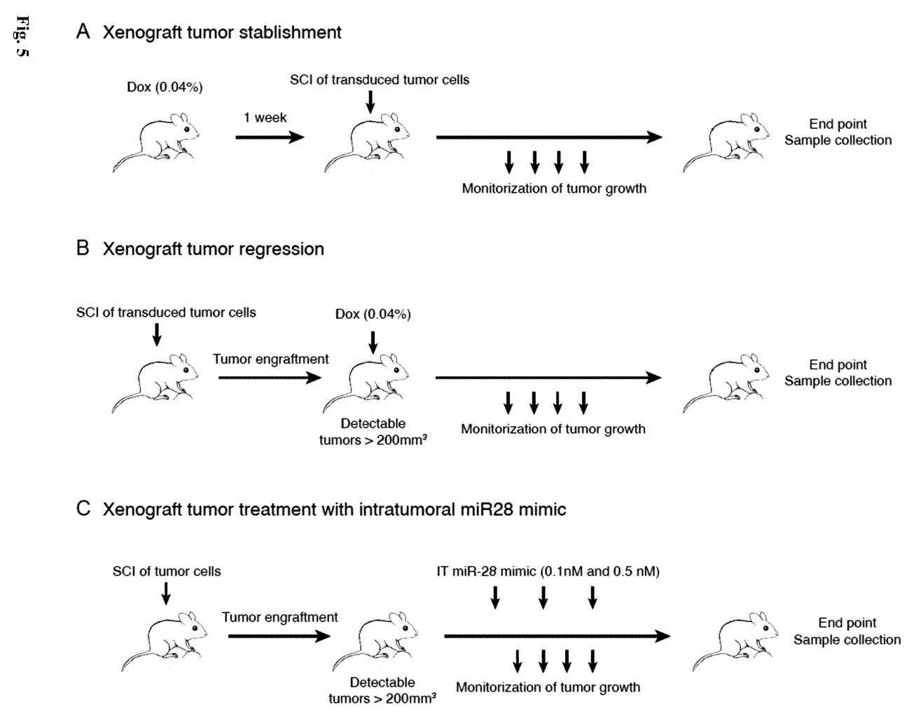

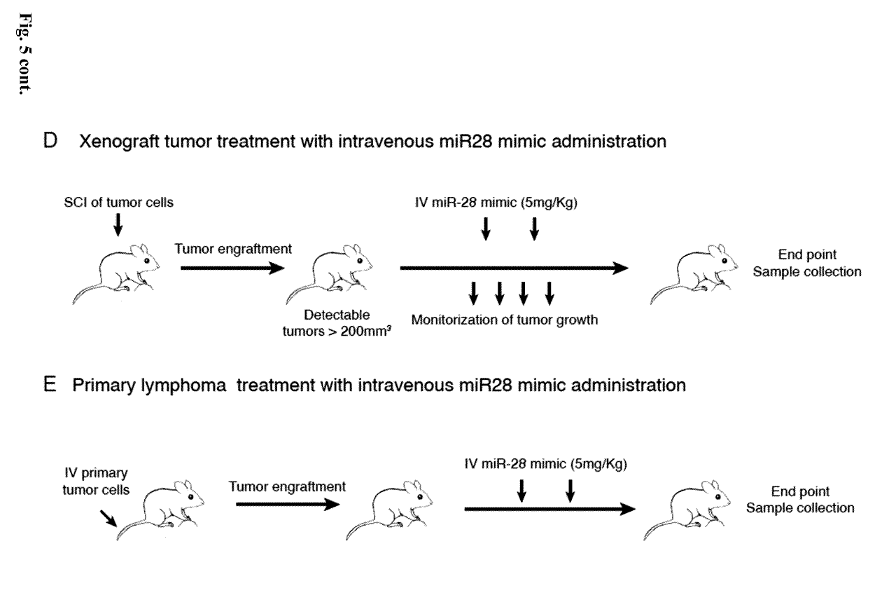

TABLE 1
miR-28 does not show toxicity in mice. Blood from mice treated as described in
FIG. 3C was analyzed for the indicated parameters. In addition, there was no evidence
of toxicity from behavioral, macroscopic or microscopic standpoints.

| | PBS | 0.5 nM control mimic | 0.5 nM miR28 mimic | Units |
|---|---|---|---|---|
| Blood Urea Nitrogen | 21.5 ± 0.70 | 23.5 ± 3.5 | 21 ± 2.82 | md/dL |
| Triglycerides | 64 ± 26.87 | 63.5 ± 3.5 | 97 ± 26.87 | md/dL |
| Total Protein | 6.3 ± 0 | 6.8 ± 0.4 | 6 ± 0.99 | md/dL |
| ALT/GPT | 102 ± 24.04 | 98 ± 21.20 | 96 ± 32.52 | U/L |
| AST/GOT | 398 ± 301.22 | 271 ± 82.0 | 259 ± 352.14 | U/L |
| GGT | 12.5 ± 3.53 | 15.5 ± 9.2 | 7 ± 7.07 | U/L |
| Total Cholesterol | 125 ± 7.21 | 105.1 ± 10.3 | 60.65 ± 32.17 | mg/dL |
| Alkaline phosphatase | 94.5 ± 47.37 | 68.5 ± 81.3 | 82 ± 16.97 | U/L |
| Glucose | 134.5 ± 16.26 | 147.5 ± 23.3 | 91 ± 0 | mg/dL |
| Albumin | 1.25 ± 0.07 | 1.25 ± 0.20 | 1.2 ± 0 | g/dL |

### Example 2

### miR-28 is a Negative Regulator of the Germinal Center Reaction

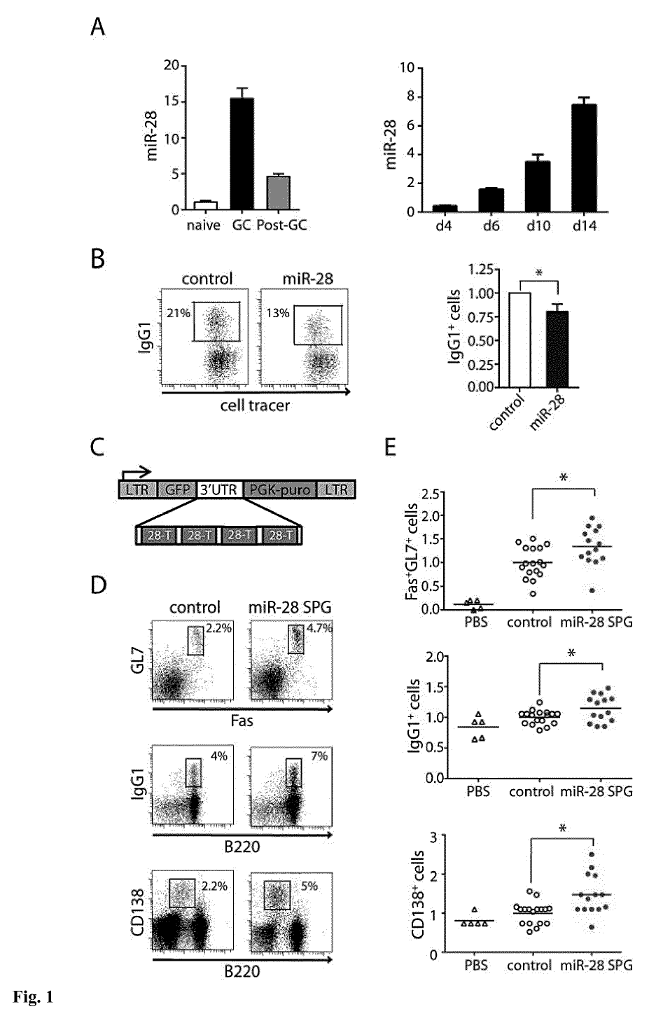

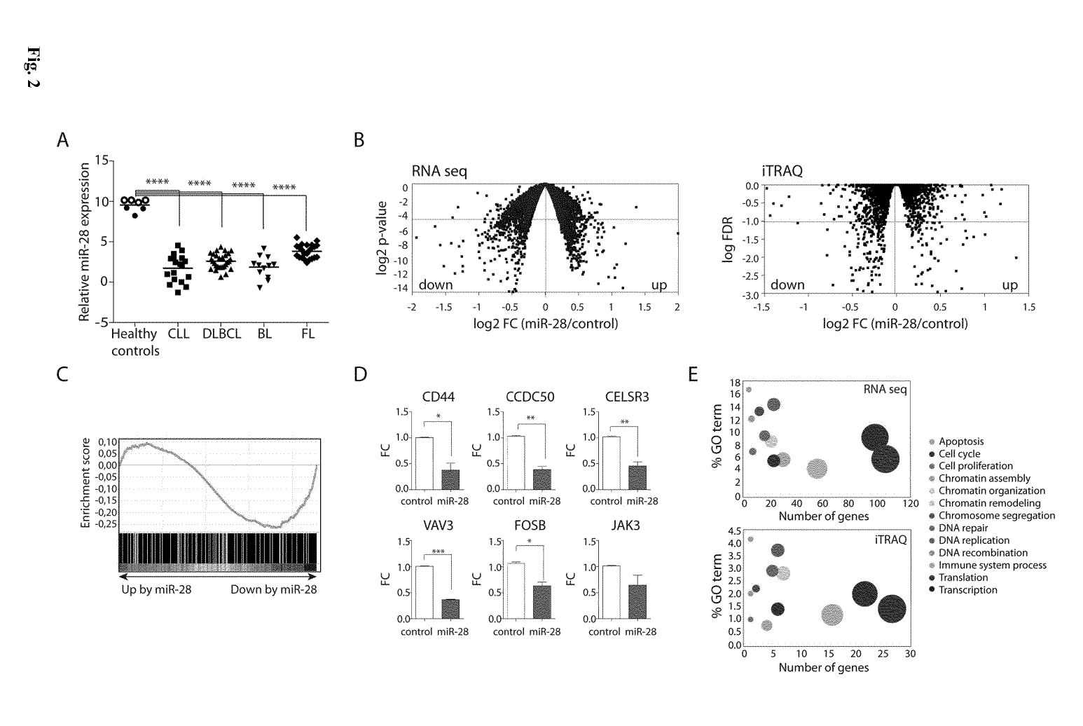

miR-28 is expressed in mouse and human germinal center (GC) B cells; however, its role in the GC reaction remains unknown. We first measured miR-28 expression by quantitative RT-PCR (qRT-PCR) in naïve B cells (CD19+Fas−GL7−), in GC B cells (CD19+Fas+GL7+) and IgA-switched post-GC B cells (CD19+Fas−GL7−IgA+) isolated from Peyer's patches and found that miR-28 expression sharply increased in GC B cells and declined soon afterwards in post-GC cells (FIG. 1A, left), reinforcing the idea that miR-28 is specifically and transiently expressed in GC B cells. Likewise miR-28 expression progressively increases during the GC reaction, as measured in splenic GC B cells of mice immunized with sheep red blood cells (SRBC) (FIG. 1A, right).

To investigate the functional relevance of miR-28 expression in GC B cells, we isolated naïve B cells from mouse spleens, cultured them in the presence of LPS and IL-4 to recapitulate events that take place during GC generation in vivo, and transduced them with miR-28 or control retroviruses. miR-28 expression promoted a mild but consistent decrease in the proportion of cells undergoing CSR (FIG. 1B). We next generated loss of function mouse models using bone marrow chimeras in which miR-28 expression was neutralized by transduction of a miR-28 sponge construct (miR-28 SPG) (FIG. 1C), which competes for binding to endogenous miR-28 binding sites. Bone marrow cells from wild type CD45.2 mice were transduced with miR-28 SPG or control retrovirus and transferred to lethally irradiated CD45.1 congenic recipients. Mice reconstituted for 4 weeks were immunized with a T cell-dependent antigen and analysed 10-14 days later. Compared with control chimeras, immunized miR-28 SPG chimeras generated significantly higher proportions of GC cells (Fas+GL7+, top panels), switched cells (IgG1+, middle panels) and plasma cells (CD138+, bottom panels) (FIG. 1D and E), indicating that miR-28 inhibition favours the GC response in vivo. These gain- and loss-of-function experiments indicate that miR-28 is a negative regulator of the GC reaction.

### Example 3

### miR-28 is Downregulated in GC Derived Neoplasms

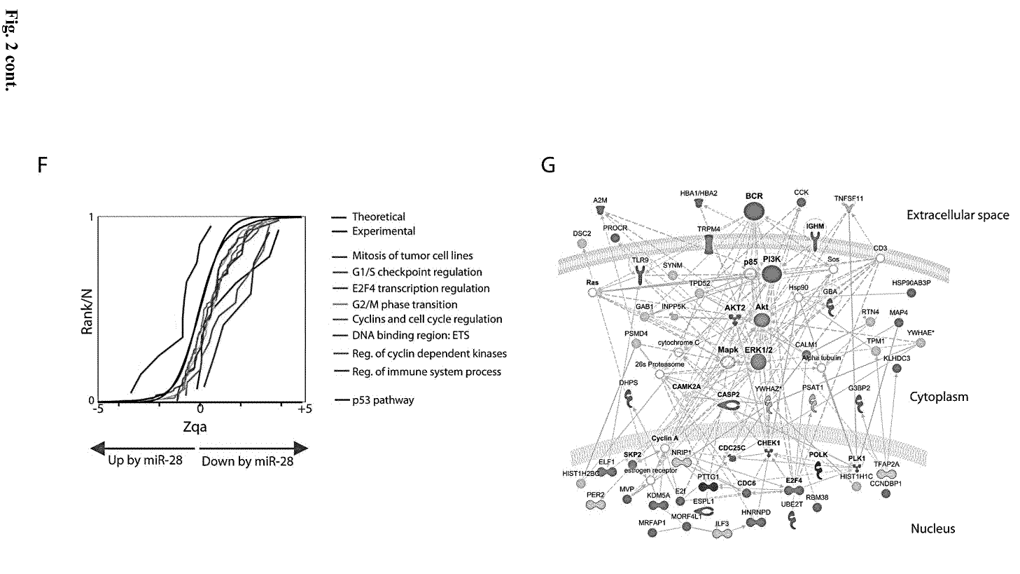

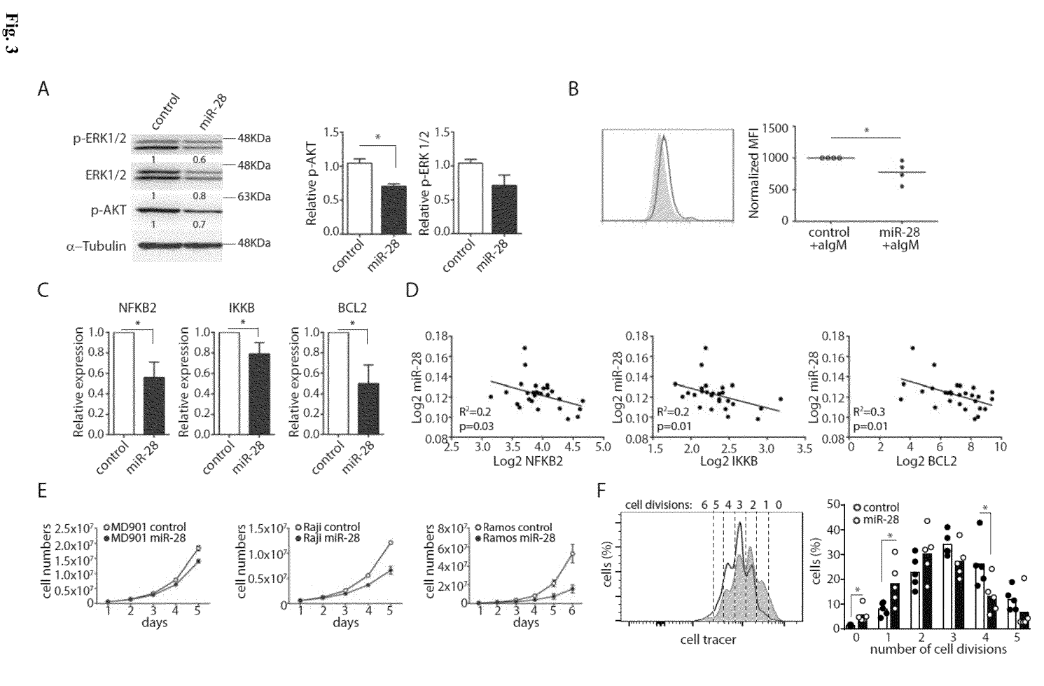

The finding that miR-28 negatively regulates the GC reaction prompted us to explore the connection between miR-28 expression and B cell lymphomagenesis. miRNA profiling in an extensive dataset of human primary GC-derived B cell neoplasms (GSE 29493 (Di Lisio et al., 2012)) revealed that miR-28 expression is lost in several GC-derived lymphoma subtypes, including BL (p=6.9×10-11), DLBCL (p=2.6×10-18), and FL (p=9.3×10-16), and CLL leukemia (p=1.7×10-10) (FIG. 2A). miR-28 downregulation is also a common event in established GC-derived B cell lines, such as the Ramos and Raji BL cell lines (p=0.002 and p=0.0012, respectively) and the MD901 DLBCL cell line (p=0.004).

### Example 4

### Transcriptome and Proteome Profiling of miR-28 Targets

To accurately identify the genes regulated by miR-28 in B cells we performed combined transcriptome and proteome analysis upon inducible re-expression of miR-28 in the human BL cell line Ramos, in which miR-28 expression is much lower than in normal GC B cells. To re-express miR-28 we transduced Ramos cells with a pTRIPZ lentiviral vector encoding the miR-28 precursor and red fluorescent protein (RFP), both inducible by doxycycline treatment. RFP+ cells were isolated by flow cytometry and subjected to RNAseq analysis for transcriptome profiling and to deep quantitative proteomics using multiplexed isobaric labeling (iTRAQ). RNAseq analysis revealed miR-28-induced expression changes in 1202 transcripts (p<0.05), 568 of which were downregulated (FIG. 2B). GSEA showed that computer-predicted miR-28 targets were significantly enriched in transcripts downregulated by miR-28 (FIG. 2C). miR-28-induced downregulation was validated by qRT-PCR for CD44, CCDC50, CELSR3, VAV3, FOSB, and JAK3 (FIG. 2D). iTRAQ analysis allowed quantification of more than 7.000 proteins, revealing miR-28-induced changes in the amounts of 277 proteins (10% FDR, p<0.0038,), 171 of which were downregulated (FIG. 2B, right panel).

Pathway enrichment analysis revealed that the miR-28-induced transcriptome and proteome changes grouped in remarkably similar cellular function pathways (FIG. 2E). Cell cycle, chromatin assembly and DNA replication were among the functions mostly enriched at both the mRNA and protein levels. Because the proteome profiling yielded fewer putative targets, we performed a more thorough threshold-free analysis of all proteome alterations using the Systems Biology Triangle analysis, a novel functional class-scoring algorithm that identifies alterations in functional categories produced by coordinated protein responses in high-throughput quantitative proteomics experiments. This analysis showed that miR-28 expression in Ramos BL cells induces the coordinated downregulation of proteins belonging to different cellular pathways linked to cell cycle progression (FIG. 2F). Finally, Ingenuity Pathway Analysis of gene networks showed that proteins whose levels are altered by miR-28 form a highly significant cluster of genes involved in BCR signaling. This gene network contains four main hubs, BCR, PI3K, AKT, and ERK1/2, which play pivotal roles in B cell biology and regulate the induction of cell cycle and apoptosis regulatory molecules such as CDC6, CDC25C, TNFSF11 and ILF3 (FIG. 2G).

These results thus suggest that reintroducing miR-28 into lymphoma cells affects the signaling network emanating from the BCR.

### Example 5

### miR-28 Dampens BCR Signaling and Impairs B Cell Proliferation and Survival

To assess the functional impact of miR-28 on BCR signaling, we quantified the phosphorylated and active forms of AKT (p-AKT) and ERK (p-ERK). BL cells transduced with miR-28 tended to have lower p-ERK levels than RFP+ control-transduced cells (FIG. 3A). Similarly, phosphorylation of p-AKT on serine 473 was significantly lower in cells expressing miR-28 (34% below control cells, p=0.02). Moreover, flow cytometry analysis of cells stimulated with anti-IgM revealed that miR-28-transduced BL cells had lower level of p-AKT than controls (FIG. 3B) (p=0.04), indicating that miR-28 expression dampens BCR signalling in BL cells.

Many B cell neoplasms are dependent on BCR signalling for proliferation and survival, and activating mutations in several BCR pathway components have been identified. The identification of BCR signalling as a main hub for miR-28 regulatory activity prompted us to examine our RNAseq data for alterations to other miR-28-regulated BCR-related pathway components, focusing on NFKB2, IKKB, and BCL2.

NFKB2 and IKKB are components of the NF-κB pathway, a major survival pathway downstream of the BCR and the most commonly altered gene pathway in lymphoid malignancies. BCL2 is an anti-apoptotic protein induced by the BCR, and genetic gain-of-function BCL2 alterations are found in various mature B cell malignancies, including CLL, FL, and DLBCL. NFKB2, IKKB and BCL2 were all downregulated in miR-28-expressing Ramos BL cells, a result confirmed by qRT-PCR (FIG. 3C). Importantly, transcript levels for NFKB2, IKKB and BCL2 correlate inversely with miR-28 expression in human primary GC-derived lymphoma subsets of the ABC DLBCL subtype (FIG. 3D), which is a subtype known to specifically rely on chronic active BCR signaling for survival. Overall, these results show that miR-28 expression downregulates downstream effectors of BCR signaling that play key roles in

B lymphocyte proliferation and survival, and whose expression is frequently upregulated in GC-derived malignancies.

To examine whether miR-28 expression regulates cell cycle and survival, we first expressed miR-28 in different human B cell lymphomas and analysed cell growth in vitro. Overexpression of miR-28 for 5-6 days reduced cell number by 40% in the MD901 ABCDLBCL cell line, and by 45% and 77% in the Raji and Ramos BL cell lines, respectively (FIG. 3E). To further expand these findings to primary B cells, we retrovirally transduced naive mouse splenic B cells with miR-28 and measured cell division. Interestingly, mouse primary B cells overexpressing miR-28 had a lower proliferation index and a higher proportion of cells that have undergone few divisions (0-2), compared to control B cells (FIG. 3F). In addition, cell cycle analysis and BrdU incorporation assays revealed that miR-28-expressing Ramos cultures contained a significantly lower proportion of proliferating cells than RFP+ control-transduced cultures (FIG. 3G-H). miR-28 reexpression in BL cells also significantly increased apoptotic cell death, as indicated by higher proportions of annexin V+ 7AAD-early apoptotic cells (2-fold increase in versus control RFP+ cells; p=0.015) and active caspase 3+ cells (3-fold increase versus control RFP+ cells; p=0.037) (FIG. 3). Together, these results show that miR-28 expression dampens BCR signalling and impairs the proliferation and survival of both primary and tumor B lymphocytes.

### Example 6

### miR-28 Suppresses Tumor Growth in GC-Derived Neoplasms

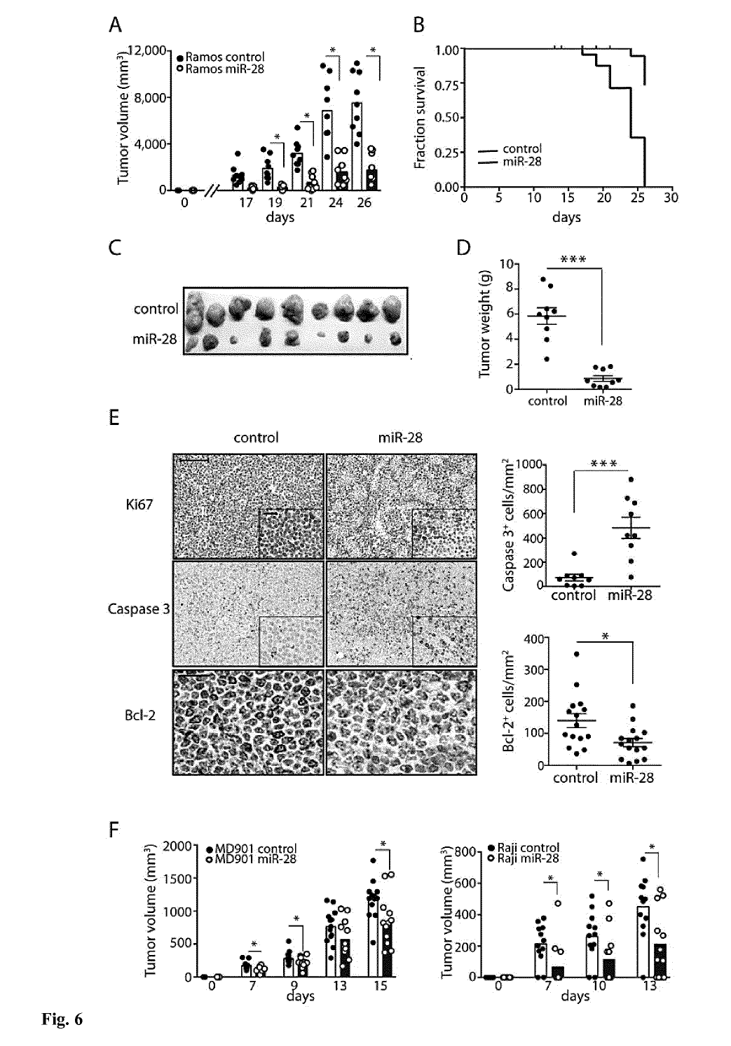

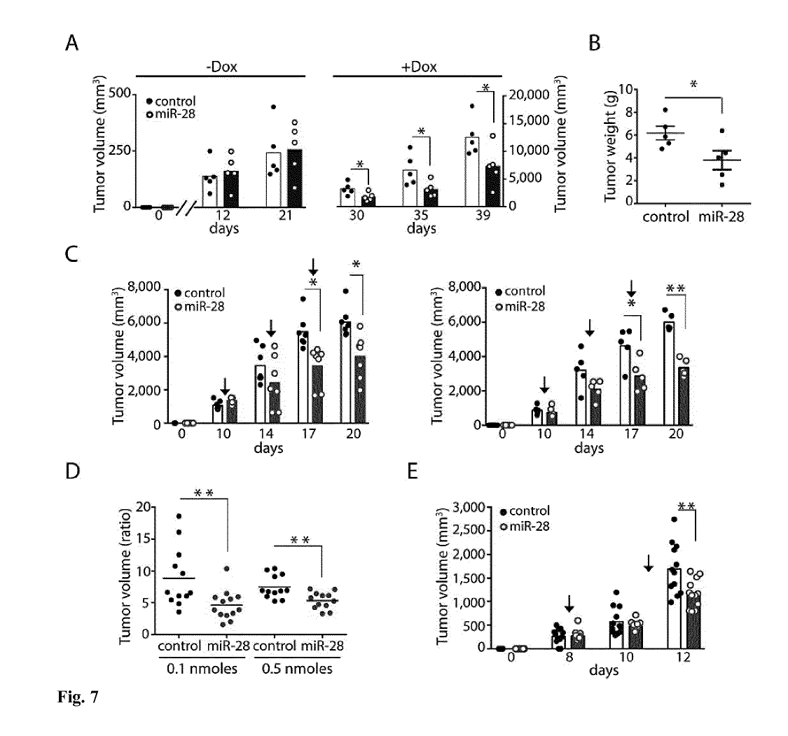

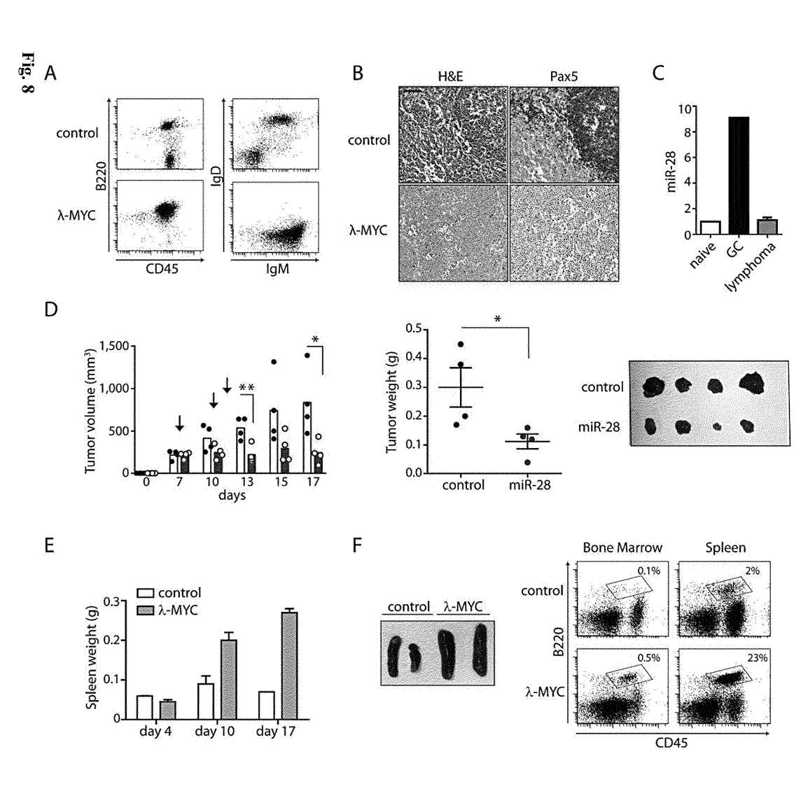

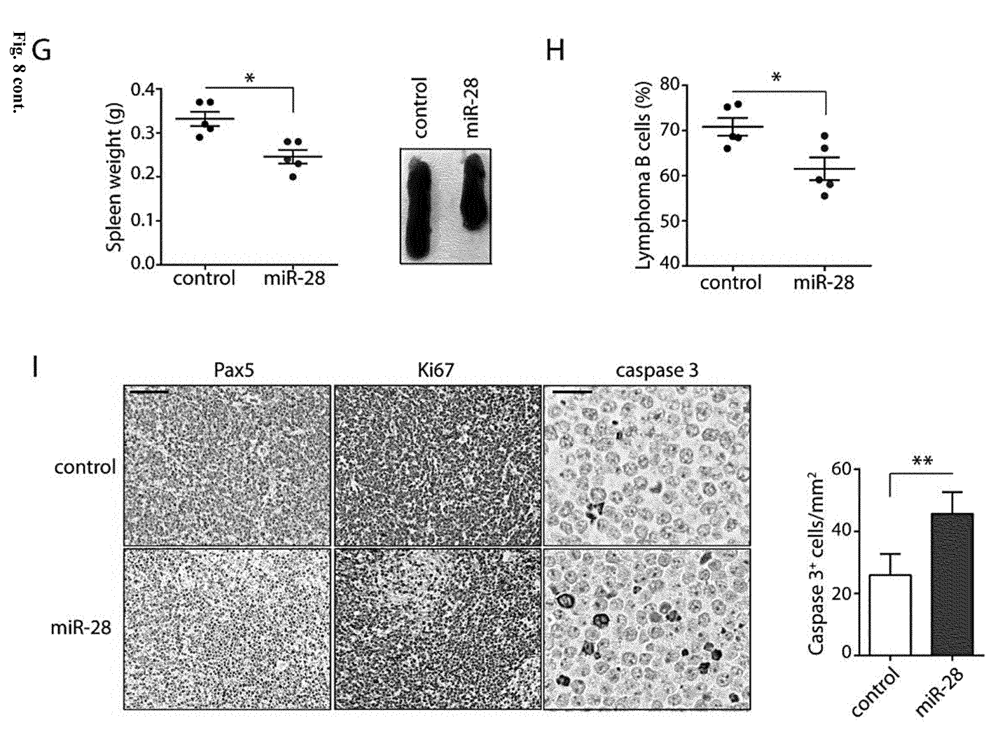

To assess the tumour suppressor activity of miR-28 in vivo we first analysed the effect of re-expression on lymphoma growth in xenograft models. Ramos cells were transduced with miR-28 or scramble pTRIPZ lentiviral vectors and induced with doxycycline, and the resulting miR-28 RFP+ or control RFP+ lymphoma cells were injected subcutaneously into either flank of NOD scid gamma (NSG) immunodeficient mice. miR-28 expression significantly slowed the growth of xenograft tumours (FIG. 6A-B), indicating that miR-28 has antitumor activity. Indeed, endpoint tumour mass was markedly lower when xenografted BL cells re-expressed miR-28 (FIG. 6C-D). Immunohistochemical analysis revealed that miR-28-expressing Ramos BL tumors also contained fewer Ki67+ proliferating cells and Bc1-2 expressing cells. Conversely, miR-28-expressing tumours had higher proportion of apoptotic caspase 3+ cells (FIG. 6E).

These findings suggest that miR-28 re-expression impairs proliferation and survival of lymphoma cells. In a further test, we repeated the miR-28 re-expression xenograft assay with an additional BL lymphoma cell line (Raji) and an ABC subtype DLBCL (MD901). Monitoring of tumor growth over 2 weeks showed that miR-28 re-expression slowed the growth of Raji and MD901 tumors (FIG. 6F), demonstrating that miR-28 antitumoral activity is not restricted to a single lymphoma type. These results show that miR-28 has tumor suppressor activity in both BL and DLBCL GC-derived lymphomas and interferes with lymphoma establishment in vivo. To determine if miR-28 can also impair the growth of established lymphomas, we injected miR-28-transduced Ramos cells into NSG mice before induction of the constructs with doxycycline. Tumours were allowed to grow until they reached a volume of 200 mm³, and only then was doxycycline administered in the drinking water, followed by monitoring of tumour growth over several weeks. We found that miR-28 replacement significantly slowed the growth rate of established Ramos BL tumours (FIG. 7A), with tumor mass 47%-58% lower than in control tumors (FIG. 7B). These results show the therapeutic potential of in vivo miR-28 delivery for the treatment of established GC-derived lymphomas.

Next, as a proof-of-concept that miR-28 replacement strategies might hold promising therapeutic potential, we assayed the anti-tumoral activity of a synthetic miR-28 mimic, an analog of the natural miRNA chemically modified to enhance stability and activity. First, wild type Ramos BL cells were injected subcutaneously into NSG mice, tumours were allowed to reach a volume of 200 mm³, and mice were then given 3 intratumor injections with a selected miR-28 mimic dose.

At both doses (0.1 or 0.5 nmol) and all time points analyzed, miR-28 mimic delayed the growth of established BL tumors (FIG. 7C-D). We next tested the efficacy of miR-28 mimic when administered intravenously. Ramos BL xenografts were established as in the previous experiment, and mice were then given 2 intravenous injections with miR-28 mimic oligonucleotides. Mice treated intravenously with miR-28 mimic harbored notably smaller tumours than mice treated with control (scramble) mimics (FIG. 6E). Histopathological analysis of kidney, liver, heart and spleen of mice treated with intravenous miR-28 mimic showed no evidence of macroscopic or microscopic tissue alterations, indicating that this treatment does not have severe toxic effects.

To study the therapeutic potential of miR-28 in primary lymphomas, we made use of the lambda/MYC transgenic (λ-MYC) mouse model, in which the Myc proto-oncogene is constitutively expressed under the regulatory elements of the immunoglobulin X light chain gene and recapitulates many pathogenic features of human BL (FIG. 8A-B). As expected, miR-28 expression was upregulated in normal GC B cells from λ-MYC mice. More remarkably, miR-28 expression in λ-MYC mice was lost upon B cell transformation, in agreement with the loss of miR-28 in numerous human B cell neoplasms (FIG. 8C). To assess the therapeutic potential of miR-28 replacement in primary BL in vivo, we first subcutaneously injected BL cells from enlarged spleens or lymph nodes of λ-MYC mice into NSG host mice. At days 7, 10 and 13 after tumour transplant, recipient mice were treated by intra-tumoral administration of miR-28 mimic. Growth was clearly reduced in tumours treated with miR-28 mimic compared with those treated with scramble mimic (FIG. 8D). These experiments were followed by a systemic approach, in which BL cells from λ-MYC mice were injected intravenously into NSG recipients. In this model, transplanted cells home to the spleen, bone marrow and lymph nodes of recipients, and spleen enlargement is detected from 10 days onward in mice receiving λ-MYC cells, compared with mice receiving cells from control littermates (FIG. 8E). Spleen enlargement correlates with a large expansion of λ-MYC B cells that is also detectable in the bone marrow and most likely reflects successful tumor engraftment (FIG. 8F). At days 10 and 14 after λ-MYC BL transplant, NSG mice were intravenously injected with miR-28 mimic, and mice were sacrificed for analysis 3 days later. The spleens of miR-28-treated mice were notably smaller than those treated with control mimic (FIG. 8G) and contained a lower proportion of BL B cells (FIG. 11H). Indeed, intravenous miR-28 mimic reduced the proportion of B lymphoma cells and proliferating Ki67+ cells in spleen and increased the numbers of caspase 3+ apoptotic cells.

Together, these results demonstrate that therapeutic strategies that promote miR-28 reexpression have the potential to impair B-cell tumor growth in vivo by diminishing lymphoma proliferation and promoting cell death.

1. A method of treating a subject suffering from a diffuse large B cell lymphoma (DLBCL), comprising administering a composition comprising a miRNA-28 mimic, a miRNA-28 expression vector and/or a miRNA-28 to the subject, wherein the nucleobase sequence of the miRNA-28 mimic has at least 80% identity with the nucleobase sequence of SEQ ID NO: 1.

2. The method of claim 1, wherein the subject suffers or has a diffuse large B cell lymphoma (DLBCL) of the activated B cell (ABC) subtype.

3. The method of claim 1, wherein the subject is a human being.

4. The method of claim 1, wherein the composition comprises the miRNA-28 mimic and the composition is administered to the subject via intravenous administration, subcutaneous administration, intratumoral administration, or chemoembolization.

5. The method of claim 4, wherein the composition is administered via intravenous administration.

6. The method of claim 1, wherein the miRNA-28 mimic compound comprises a complementary oligonucleotide hybridized to the oligonucleotide.

7. The method of claim 1, wherein the composition comprises at least one lipid.

8. The method of claim 7, wherein the composition comprises a cationic lipid, a neutral lipid, a sterol and a disaggregation lipid.

9. The method of claim 1, wherein the miRNA-28 expression vector is a lentiviral vector.

10. The method of claim 9, wherein the expression vector comprises SEQ ID NO 2.

11. The method of claim 1, wherein the miRNA-28 mimic has a 100% identity with the nucleobase sequence of SEQ ID NO 1.


SEQUENCE LISTING (4 sequences)

SEQ ID NO: 1 - RNA, 86 nt, Artificial Sequence
  Synthetic miR-28 mimic
  misc_feature (1)..(86)
gguccuugcccucaaggagcucacagucuauugaguuaccuuucugacuuucccacuagauugugagcuccuggagggcaggcacu

SEQ ID NO: 2 - RNA, 86 nt, Artificial Sequence; identical to SEQ ID NO: 1
  Synthetic miR-28 mimic
  misc_feature (1)..(86)

SEQ ID NO: 3 - DNA, 83 bp, Artificial Sequence
  Forward partially complementary oligonucleotide for the generatioin of retroviral constructs.
  misc_feature (1)..(83)
tcgactagctagaacgcggccgcctcaatagactgtgagctccttcgatctcaatagactgtgagctccttaccggtctcaat

SEQ ID NO: 4 - DNA, 84 bp, Artificial Sequence
  Reverse partially complementary sequence for generating retroviral constructs.
  misc_feature (1)..(84)
gaagtcagtccgattctcgagaaggagctcacagtctattgaggtgaaaggagctcacagtctattgagaccggtaaggagctc